(19) United States
(12) Patent Application Publication
(10) Pub. No.: US 2022/0265121 A1
(43) Pub. Date: Aug. 25, 2022

(54) SYSTEMS AND METHODS FOR CONTROLLING A SURGICAL PUMP USING ENDOSCOPIC VIDEO DATA

(71) Applicant: Stryker Corporation, Kalamazoo, MI (US)

(72) Inventors: Brian FOUTS, Morgan Hill, CA (US); Cole Kincaid HUNTER, Santa Clara, CA (US); Brady WOOLFORD, Mapleton, UT (US); Amit A. MAHADIK, San Jose, CA (US); Hannes RAU, Milpitas, CA (US); Wenjing LI, Sunnyvale, CA (US); Joel M. ERNST, San Jose, CA (US)

(73) Assignee: Stryker Corporation, Kalamazoo, MI (US)

(21) Appl. No.: 17/651,412

(22) Filed: Feb. 16, 2022

Related U.S. Application Data
(60) Provisional application No. 63/153,857, filed on Feb. 25, 2021.

(51) Int. Cl. A61B 1/00; G06T 7/00; G06V 10/764; A61B 1/317; A61B 1/015; A61B 34/20

(52) U.S. Cl. CPC A61B 1/00006; G06T 7/0012; G06V 10/764; A61B 1/317; A61B 1/000096; A61B 1/000094; A61B 1/015; A61B 34/20; G06T 2207/10016; G06T 2207/20081; G06T 2207/10068; A61B 2034/2055

(57) ABSTRACT

According to an aspect, video data taken from an endoscopic imaging device can be used to automatically control a surgical pump for purposes of regulating fluid pressure in an internal area of a patient during an endoscopic procedure. Control of the pump can be based in part on one or more features extracted from video data received from an endoscopic imaging device. The features can be extracted from the video data using a combination of machine learning classifiers and other processes configured to determine the presence of various conditions within the images of the internal area of the patient. Using the one or more extracted features, the system can adjust the inflow and outflow settings of the surgical pump to regulate the fluid pressure of the internal area of the patient commensurate with the needs of the surgery and the patient at any given moment in time during the surgical procedure.

### CROSS-REFERENCE TO RELATED APPLICATIONS

This application claims the benefit of U.S. Provisional Application No. 63/153,857, filed Feb. 25, 2021, the entire contents of which are hereby incorporated by reference herein.

### FIELD

This disclosure relates to controlling an arthroscopy fluid pump configured to irrigate an internal area of a patient during a minimally invasive surgical procedure, and more specifically, to using video data taken from an endoscopic imaging device to automatically control the amount and pressure of fluid pumped into the internal area of the patient.

### BACKGROUND

Minimally invasive surgery generally involves the use of a high-definition camera coupled to an endoscope inserted into a patient to provide a surgeon with a clear and precise view within the body. When the endoscope is inserted into the internal area of a patient's body prior to or during a minimally invasive surgery, it is important to maintain an environment within the internal area that is conducive to clearly visualizing the area by the camera. For instance, keeping the internal area clear of blood, debris, or other visual impairments are critical to ensuring that a surgeon or other practitioner has adequate visibility of the internal area.

One way to keep an internal area relatively free and clear of visual disturbances during an endoscopic procedure is to irrigate the internal area with a clear fluid such as saline during the procedure. Irrigation involves introducing a clear fluid into the internal area at a particular rate (i.e., inflow), and removing the fluid by suction (i.e., outflow) such that a desired fluid pressure is maintained in the internal area. The constant flow of fluid can serve two purposes. First, the constant flow of fluid through the internal area of the patient can help to remove debris from the field of view of the imaging device, as the fluid carries the debris away from the area and is subsequently suctioned out of the area. Second, the fluid creates a pressure build up in the internal area which works to suppress bleeding by placing pressure on blood vessels in or around the internal area.

Irrigating an internal area during a minimally invasive surgery comes with risks. Applying too much pressure to a joint or other internal area of a patient can cause injury to the patient and can even permanently damage the area. Thus, during an endoscopic procedure, the fluid delivered to an internal area is managed to ensure that the pressure is high enough to keep the internal area clear for visualization, but low enough so as to not cause the patient harm. Surgical pumps can be utilized to perform fluid management during an endoscopic procedure. Surgical pumps regulate the inflow and outflow of irrigation fluid to maintain a particular pressure inside an internal area being visualized. The surgical pump can be configured to allow for the amount of pressure to be applied to an internal area to be adjusted during a surgery.

The amount of pressure needed during a surgery can be dynamic depending on a variety of factors. For instance the amount of pressure to be delivered can be based on the joint being operated on, the amount of bleeding in the area, as well the absence or presence of other instruments. Having the surgeon manually manage fluid pressure during a surgery can place a substantial cognitive burden on them. The surgeon has to ensure that the pump is creating enough pressure to allow for visualization of the internal area, while simultaneously minimizing the pressure in the internal area so as to prevent injury or permanent damage to the patient. In an environment where the pressure needs are constantly changing based on conditions during the operation, the surgeon will have to constantly adjust the pressure settings of the pump to respond to the changing conditions. These constant adjustments can be distracting, and reduce the amount of attention that the surgeon has towards the actual procedure itself.

### SUMMARY

According to an aspect, video data taken from an endoscopic imaging device can be used to automatically control a surgical pump for purposes of regulating fluid pressure in an internal area of a patient during an endoscopic procedure. In one or more examples, control of the pump can be based in part on one or more features extracted from video data received from an endoscopic imaging device. The features can be extracted from the video data using a combination of machine learning classifiers and other processes configured to determine the presence of various conditions within the images of the internal area of the patient. Optionally, the machine learning classifiers can be configured to determine the anatomy displayed in a particular image as well as the procedure step shown in a given image. Using these two determinations, the systems and methods described herein can adjust the inflow and outflow settings of the surgical pump to regulate the fluid pressure of the internal area of the patient commensurate with the needs of the surgery and the patient at any given moment in time during the surgical procedure. Optionally, the machine learning classifiers can be configured to determine the presence of an instrument in the internal area. Based on the determination, the surgical pump can be controlled to adjust the pressure settings or can also switch the source of suction from a dedicated device to another device depending on what instruments are determined to be present in the internal area.

According to an aspect, the surgical pump can be controlled based on one or more image clarity classifiers. In one or more examples, one or more machine learning classifiers and/or algorithms can be applied to receive video data to determine one or more characteristics associated with the clarity of the video. If the clarity of the video is determined to be inadequate, the system and methods described herein can be configured to adjust the surgical pump in a manner that will improve the quality of the video, while also minimizing the risk of the patient becoming injured or suffering permanent damage as a result of too much pressure applied by the pump. Optionally, the one or more algorithms to determine image clarity can include algorithms configured to detect blood, debris, snow globe conditions, turbidity that are present in the video data. In one or more examples, the algorithms to determine clarity of the video can include changing the color space of received video data to a color space that may be more conducive to artifact detection by the algorithm.

According to an aspect, a method for controlling a fluid pump for use in surgical procedure includes: receiving video data captured from an imaging tool configured to image an internal portion of a patient, applying one or more machine learning classifiers to the received video data to generate one or more classification metrics based on the received video data, wherein the one or more machine learning classifiers are created using a supervised training process that comprises using one or more annotated images to train the machine learning classifier, determining the presence of one or more conditions in the received video data based on the generated one or more classification metrics, and determining an adjusted setting for the flow through or head pressure from the fluid pump based on the determined presence of the one or more conditions in the received video data. The method can include adjusting the flow through or head pressure from the fluid pump based on the determined presence of the one or more conditions in the received video data. The imaging tool can be pre-inserted into the internal portion of the patient.

Optionally, the supervised training process includes: applying one or more annotations to each image of a plurality of images to indicate one or more conditions associated with the image, and processing each image of the plurality of images and its corresponding one or more annotations.

Optionally, the one or more machine learning classifiers comprises a joint type machine learning classifier configured to generate one or more classification metrics associated with identifying a type of joint pictured in the received video data.

Optionally, the joint type machine learning classifier is trained using one or more training images, each training image annotated with a type of joint pictured in the training image.

Optionally, the joint type machine learning classifier is configured to identify one or more joints selected from the group consisting of a hip, a shoulder, a knee, an ankle, a wrist, and an elbow.

Optionally, the joint type machine learning classifier is configured to generate one or more classification metrics associated with identifying whether the imaging tool is not within a joint.

Optionally, the one or more machine learning classifiers include a procedure stage machine learning classifier configured to generate one or more classification metrics associated with identifying a procedure stage being performed in the received video data.

Optionally, the procedure stage machine learning classifier is trained using one or more training images, each training image annotated with a stage of a surgical procedure pictured in the training image.

Optionally, adjusting the flow through or head pressure from the fluid pump comprises adjusting one or more settings of the fluid pump.

Optionally, adjusting one or more settings of the fluid pump based on the determined presence of the one or more conditions in the received video data comprises adjusting a pressure setting of the fluid pump based on the generated classification metrics associated with the joint type machine learning classifier and the procedure stage machine learning classifier.

Optionally, adjusting one or more settings of the fluid pump based on the determined presence of the one or more conditions in the received video data comprises adjusting a flow setting of the fluid pump based on the generated classification metrics associated with the joint type machine learning classifier and the procedure stage machine learning classifier.

Optionally, the one or more machine learning classifiers comprises an instrument identification machine classifier configured to generate one or more classification metrics associated with identifying one or more instruments in the received video data.

Optionally, the instrument identification machine learning classifier is trained using one or more training images annotated with a type of instrument pictured in the training image.

Optionally, the instrument identification machine classifier is configured to identify instruments selected from the group consisting of a shaver tool, a radio frequency (RF) probe, and a dedicated suction device.

Optionally, the fluid pump is configured to activate a suction functionality of the one or more instruments based on the one or more classification metrics generated by the instrument identification machine classifier.

Optionally, the one or more machine learning classifiers include an image clarity machine learning classifier configured to generate one or more classification metrics associated with a clarity of the received video data.

Optionally, the image clarity machine classifier is configured to generate one or more classification metrics associated with an amount of blood visible in the received video data.

Optionally, the image clarity machine classifier is configured to generate one or more classification metrics associated with an amount of bubbles visible in the received video data.

Optionally, the image clarity machine classifier is configured to generate one or more classification metrics associated with an amount of debris visible in the received video data.

Optionally, the image clarity machine classifier is configured to generate one or more classification metrics associated with whether the internal portion of a patient being imaged has collapsed.

Optionally, determining the presence of one or more conditions in the received video data based on the generated one or more classification metrics comprises determining if a clarity of the video is above a pre-determined threshold, and wherein the determination is based on the one more classification metrics generated by the image clarity machine classifier.

Optionally, if it is determined that the clarity of the video is below the pre-determined threshold, determining if the fluid pump is operating at a maximum allowable pressure setting.

Optionally, if it is determined that the fluid pump is not operating at the maximum allowable pressure setting, increasing a pressure setting of the fluid pump.

Optionally, wherein if it is determined that the clarity of the video is above the pre-determined threshold, determining if the fluid pump is operating above a minimum allowable pressure setting.

Optionally, if it is determined that the fluid pump is operating above the minimum allowable pressure setting, decreasing a pressure setting of the fluid pump.

Optionally, the fluid pump is for fluid inflow to the internal portion of the patient.

Optionally, the fluid pump is for fluid outflow from the internal portion of the patient.

According to an aspect, a method for controlling a fluid pump for use in surgical procedures includes receiving video data captured from an imaging tool configured to image an internal portion of a patient, detecting disturbances within the received video data by identifying one or more visual characteristics in the received video, creating a plurality of classification metrics for classifying disturbances in the video data, determining the presence of one or more conditions in the received video data based on the plurality of classification metrics and the one or more visual characteristics, and determining an adjusted setting for the flow through or head pressure from the fluid pump based on the determined presence of the one or more conditions in the received video data. The method can include adjusting the flow through or head pressure from the fluid pump based on the determined presence of the one or more conditions in the received video data.

Optionally, adjusting the flow through or head pressure from the fluid pump comprises adjusting one or more settings of the fluid pump.

Optionally, the method comprises capturing one or more image frames from the received video data, and detecting disturbances within the received video data comprises detecting disturbances within each captured image frame of the one or more image frames.

Optionally, detecting disturbances within the received video data comprises detecting an amount of blood in a frame of the received video.

Optionally, detecting an amount of blood in a frame of the received video includes identifying one or more bleed regions in the frame of the received video data, identifying a total imaged area in the frame of the received video data, calculating an area of each identified bleed region, calculating a ratio of a sum of the calculated areas of each identified bleed region over the total imaged area in the frame of the received video data, and comparing the calculated ratio with a pre-determined threshold.

Optionally, detecting an amount of blood in a frame of the received video comprises converting a color space of a frame of the received video data to a hue, saturation, value (HSV) color space.

Optionally, if the calculated ratio is greater than the pre-determined threshold, increasing a pressure setting of the fluid pump.

Optionally, detecting disturbances within the received video data comprises detecting the amount of debris in a frame of the received video.

Optionally, detecting the amount of debris in a frame of the received video includes identifying one or more pieces of debris in the frame of the received video data, determining the total number of pieces of debris identified in the received video data, and comparing the determined total number of pieces of debris identified in the received video data with a pre-determined threshold.

Optionally, identifying one or more pieces of debris in the frame of the received video data comprises applying a mean shift clustering process to the frame of the received video data and extracting one or more maximal regions generated by the means shift clustering process.

Optionally, detecting the amount of debris in a frame of the received video comprises converting a color space of a frame of the received video data to a hue, saturation, value (HSV) color space.

Optionally, wherein if the determined total number of pieces of debris identified in the received video data is greater than the pre-determined threshold, increasing a pressure setting of the fluid pump.

Optionally, the one or more disturbance detection processes comprises detecting a snow globe effect in a frame of the received video.

Optionally, detecting a snow globe effect includes identifying one or more snowy area regions in the frame of the received video data, identifying a total imaged area in the frame of the received video data, calculating an area of each identified snowy area region, calculating a ratio of a sum of the calculated areas of each identified snowy area region over the total imaged area in the frame of the received video data, and comparing the calculated ratio with a pre-determined threshold.

Optionally, wherein detecting a snow globe effect comprises converting a color space of a frame of the received video data to a hue, saturation, value (HSV) color space.

Optionally, if the calculated ratio is greater than the pre-determined threshold, increasing a pressure setting of the fluid pump.

Optionally, if the calculated ratio is greater than the pre-determined threshold, increasing the fluid suction from a shaver tool located in the internal portion of the patient.

Optionally, detecting disturbances within the received video data comprises detecting turbidity in a frame of the received video.

Optionally, detecting turbidity in a frame of the received video includes applying a Laplacian of Gaussian kernel process to the frame of the received video, calculating a blur score based on the application of the Laplacian of Gaussian kernel process to the frame of the received video, and comparing the calculated blur score with a pre-determined threshold.

Optionally, if the calculated blur score is greater than the pre-determined threshold, increasing a pressure setting of the fluid pump.

Optionally, detecting turbidity in a frame of the received video comprises converting a color space of a frame of the received video data to a gray color space.

Optionally, the fluid pump is for fluid inflow to the internal portion of the patient.

Optionally, the fluid pump is for fluid outflow from the internal portion of the patient.

According to an aspect, a system for controlling a fluid pump for use in surgical procedures includes a memory, one or more processors, wherein the memory stores one or more programs that when executed by the one or more processors, cause the one or more processors to receive video data captured from an imaging tool configured to image an internal portion of a patient, apply one or more machine learning classifiers to the received video data to generate one or more classification metrics based on the received video data, wherein the one or more machine learning classifiers are created using a supervised training process that comprises using one or more annotated images to train the machine learning classifier, determine the presence of one or more conditions in the received video data based on the generated one or more classification metrics, and adjust the flow through or head pressure from the fluid pump based on the determined presence of the one or more conditions in the received video data.

Optionally, the supervised training process includes: applying one or more annotations to each image of a plurality of images to indicate one or more conditions associated with the image, and processing each image of the plurality of images and its corresponding one or more annotations.

Optionally, the one or more machine learning classifiers comprises a joint type machine learning classifier configured to generate one or more classification metrics associated with identifying a type of joint pictured in the received video data.

Optionally, the joint type machine learning classifier is trained using one or more training images, each training image annotated with a type of joint pictured in the training image.

Optionally, the joint type machine learning classifier is configured to identify one or more joints selected from the group consisting of a hip, a shoulder, a knee, an ankle, a wrist, and an elbow.

Optionally, the joint type machine learning classifier is configured to generate one or more classification metrics associated with identifying whether the imaging tool is not within a joint.

Optionally, the one or more machine learning classifiers include a procedure stage machine learning classifier configured to generate one or more classification metrics associated with identifying a procedure stage being performed in the received video data.

Optionally, the procedure stage machine learning classifier is trained using one or more training images, each training image annotated with a stage of a surgical procedure pictured in the training image.

Optionally, adjusting the flow through or head pressure from the fluid pump comprises adjusting one or more settings of the fluid pump.

Optionally, adjusting one or more settings of the fluid pump based on the determined presence of the one or more conditions in the received video data comprises adjusting a pressure setting of the fluid pump based on the generated classification metrics associated with the joint type machine learning classifier and the procedure stage machine learning classifier.

Optionally, adjusting one or more settings of the fluid pump based on the determined presence of the one or more conditions in the received video data comprises adjusting a flow setting of the fluid pump based on the generated classification metrics associated with the joint type machine learning classifier and the procedure stage machine learning classifier.

Optionally, the one or more machine learning classifiers comprises an instrument identification machine classifier configured to generate one or more classification metrics associated with identifying one or more instruments in the received video data.

Optionally, the instrument identification machine learning classifier is trained using one or more training images annotated with a type of instrument pictured in the training image.

Optionally, the instrument identification machine classifier is configured to identify instruments selected from the group consisting of a shaver tool, a radio frequency (RF) probe, and a dedicated suction device.

Optionally, the fluid pump is configured to activate a suction functionality of the one or more instruments based on the one or more classification metrics generated by the instrument identification machine classifier.

Optionally, the one or more machine learning classifiers include an image clarity machine learning classifier configured to generate one or more classification metrics associated with a clarity of the received video data.

Optionally, the image clarity machine classifier is configured to generate one or more classification metrics associated with an amount of blood visible in the received video data.

Optionally, the image clarity machine classifier is configured to generate one or more classification metrics associated with an amount of bubbles visible in the received video data.

Optionally, the image clarity machine classifier is configured to generate one or more classification metrics associated with an amount of debris visible in the received video data.

Optionally, the image clarity machine classifier is configured to generate one or more classification metrics associated with whether the internal portion of a patient being imaged has collapsed.

Optionally, determining the presence of one or more conditions in the received video data based on the generated one or more classification metrics comprises determining if a clarity of the video is above a pre-determined threshold, and wherein the determination is based on the one more classification metrics generated by the image clarity machine classifier.

Optionally, if it is determined that the clarity of the video is below the pre-determined threshold, determining if the fluid pump is operating at a maximum allowable pressure setting.

Optionally, if it is determined that the fluid pump is not operating at the maximum allowable pressure setting, increasing a pressure setting of the fluid pump.

Optionally, wherein if it is determined that the clarity of the video is above the pre-determined threshold, determining if the fluid pump is operating above a minimum allowable pressure setting.

Optionally, if it is determined that the fluid pump is operating above the minimum allowable pressure setting, decreasing a pressure setting of the fluid pump.

Optionally, the fluid pump is for fluid inflow to the internal portion of the patient.

Optionally, the fluid pump is for fluid outflow from the internal portion of the patient.

According to an aspect, a system for controlling a fluid pump for use in surgical procedures includes a memory, one or more processors, wherein the memory stores one or more programs that when executed by the one or more processors, cause the one or more processors to: receive video data captured from an imaging tool configured to image an internal portion of a patient, detect disturbances within the received video data by identifying one or more visual characteristics in the received video, create a plurality of classification metrics for classifying disturbances in the video data, determine the presence of one or more conditions in the received video data based on the plurality of classification metrics and the one or more visual characteristics, and adjust the flow through or head pressure from the fluid pump based on the determined presence of the one or more conditions in the received video data.

Optionally, adjusting the flow through or head pressure from the fluid pump comprises adjusting one or more settings of the fluid pump.

Optionally, the processer is caused to capture one or more image frames from the received video data, and detecting disturbances within the received video data comprises detecting disturbances within each captured image frame of the one or more image frames.

Optionally, detecting disturbances within the received video data comprises detecting an amount of blood in a frame of the received video.

Optionally, detecting an amount of blood in a frame of the received video includes identifying one or more bleed regions in the frame of the received video data, identifying a total imaged area in the frame of the received video data, calculating an area of each identified bleed region, calculating a ratio of a sum of the calculated areas of each identified bleed region over the total imaged area in the frame of the received video data, and comparing the calculated ratio with a pre-determined threshold.

Optionally, detecting an amount of blood in a frame of the received video comprises converting a color space of a frame of the received video data to a hue, saturation, value (HSV) color space.

Optionally, if the calculated ratio is greater than the pre-determined threshold, increasing a pressure setting of the fluid pump.

Optionally, detecting disturbances within the received video data comprises detecting the amount of debris in a frame of the received video.

Optionally, detecting the amount of debris in a frame of the received video includes identifying one or more pieces of debris in the frame of the received video data, determining the total number of pieces of debris identified in the received video data, and comparing the determined total number of pieces of debris identified in the received video data with a pre-determined threshold.

Optionally, identifying one or more pieces of debris in the frame of the received video data comprises applying a mean shift clustering process to the frame of the received video data and extracting one or more maximal regions generated by the means shift clustering process.

Optionally, detecting the amount of debris in a frame of the received video comprises converting a color space of a frame of the received video data to a hue, saturation, value (HSV) color space.

Optionally, if the determined total number of pieces of debris identified in the received video data is greater than the pre-determined threshold, increasing a pressure setting of the fluid pump.

Optionally, the one or more disturbance detection processes comprises detecting a snow globe effect in a frame of the received video.

Optionally, detecting a snow globe effect includes identifying one or more snowy area regions in the frame of the received video data, identifying a total imaged area in the frame of the received video data, calculating an area of each identified snowy area region, calculating a ratio of a sum of the calculated areas of each identified snowy area region over the total imaged area in the frame of the received video data, and comparing the calculated ratio with a pre-determined threshold.

Optionally, detecting a snow globe effect comprises converting a color space of a frame of the received video data to a hue, saturation, value (HSV) color space.

Optionally, if the calculated ratio is greater than the pre-determined threshold, increasing a pressure setting of the fluid pump.

Optionally, if the calculated ratio is greater than the pre-determined threshold, increasing the fluid suction from a shaver tool located in the internal portion of the patient.

Optionally, detecting disturbances within the received video data comprises detecting turbidity in a frame of the received video.

Optionally, detecting turbidity in a frame of the received video includes applying a Laplacian of Gaussian kernel process to the frame of the received video, calculating a blur score based on the application of the Laplacian of Gaussian kernel process to the frame of the received video, and comparing the calculated blur score with a pre-determined threshold.

Optionally, if the calculated blur score is greater than the pre-determined threshold, increasing a pressure setting of the fluid pump.

Optionally, detecting turbidity in a frame of the received video comprises converting a color space of a frame of the received video data to a gray color space.

Optionally, the fluid pump is for fluid inflow to the internal portion of the patient.

Optionally, the fluid pump is for fluid outflow from the internal portion of the patient.

According to an aspect, a non-transitory computer readable storage medium storing one or more programs for controlling a fluid pump for use in surgical procedures, for execution by one or more processors of an electronic device that when executed by the device, cause the device to receive video data captured from an imaging tool configured to image an internal portion of a patient, apply one or more machine learning classifiers to the received video data to generate one or more classification metrics based on the received video data, wherein the one or more machine learning classifiers are created using a supervised training process that comprises using one or more annotated images to train the machine learning classifier, determine the presence of one or more conditions in the received video data based on the generated one or more classification metrics, and adjust the flow through or head pressure from the fluid pump based on the determined presence of the one or more conditions in the received video data.

In one or more examples, a computer program product is provided comprising instructions which, when executed by one or more processors of an electronic device, cause the device to receive video data captured from an imaging tool configured to image an internal portion of a patient, apply one or more machine learning classifiers to the received video data to generate one or more classification metrics based on the received video data, wherein the one or more machine learning classifiers are created using a supervised training process that comprises using one or more annotated images to train the machine learning classifier, determine the presence of one or more conditions in the received video data based on the generated one or more classification metrics, and determine an adjusted setting for the flow through or head pressure from the fluid pump based on the determined presence of the one or more conditions in the received video data. The computer program product may comprise instructions to cause the device to adjust the flow through or head pressure from the fluid pump based on the determined presence of the one or more conditions in the received video data.

Optionally, the supervised training process includes: applying one or more annotations to each image of a plurality of images to indicate one or more conditions associated with the image, and processing each image of the plurality of images and its corresponding one or more annotations.

Optionally, the one or more machine learning classifiers comprises a joint type machine learning classifier configured to generate one or more classification metrics associated with identifying a type of joint pictured in the received video data.

Optionally, the joint type machine learning classifier is trained using one or more training images, each training image annotated with a type of joint pictured in the training image.

Optionally, the joint type machine learning classifier is configured to identify one or more joints selected from the group consisting of a hip, a shoulder, a knee, an ankle, a wrist, and an elbow.

Optionally, the joint type machine learning classifier is configured to generate one or more classification metrics associated with identifying whether the imaging tool is not within a joint.

Optionally, the one or more machine learning classifiers include a procedure stage machine learning classifier configured to generate one or more classification metrics associated with identifying a procedure stage being performed in the received video data.

Optionally, the procedure stage machine learning classifier is trained using one or more training images, each training image annotated with a stage of a surgical procedure pictured in the training image.

Optionally, adjusting the flow through or head pressure from the fluid pump comprises adjusting one or more settings of the fluid pump.

Optionally, adjusting one or more settings of the fluid pump based on the determined presence of the one or more conditions in the received video data comprises adjusting a pressure setting of the fluid pump based on the generated classification metrics associated with the joint type machine learning classifier and the procedure stage machine learning classifier.

Optionally, adjusting one or more settings of the fluid pump based on the determined presence of the one or more conditions in the received video data comprises adjusting a flow setting of the fluid pump based on the generated classification metrics associated with the joint type machine learning classifier and the procedure stage machine learning classifier.

Optionally, the one or more machine learning classifiers comprises an instrument identification machine classifier configured to generate one or more classification metrics associated with identifying one or more instruments in the received video data.

Optionally, the instrument identification machine learning classifier is trained using one or more training images annotated with a type of instrument pictured in the training image.

Optionally, the instrument identification machine classifier is configured to identify instruments selected from the group consisting of a shaver tool, a radio frequency (RF) probe, and a dedicated suction device.

Optionally, the fluid pump is configured to activate a suction functionality of the one or more instruments based on the one or more classification metrics generated by the instrument identification machine classifier.

Optionally, the one or more machine learning classifiers include an image clarity machine learning classifier configured to generate one or more classification metrics associated with a clarity of the received video data.

Optionally, the image clarity machine classifier is configured to generate one or more classification metrics associated with an amount of blood visible in the received video data.

Optionally, the image clarity machine classifier is configured to generate one or more classification metrics associated with an amount of bubbles visible in the received video data.

Optionally, the image clarity machine classifier is configured to generate one or more classification metrics associated with an amount of debris visible in the received video data.

Optionally, the image clarity machine classifier is configured to generate one or more classification metrics associated with whether the internal portion of a patient being imaged has collapsed.

Optionally, determining the presence of one or more conditions in the received video data based on the generated one or more classification metrics comprises determining if a clarity of the video is above a pre-determined threshold, and wherein the determination is based on the one more classification metrics generated by the image clarity machine classifier.

Optionally, if it is determined that the clarity of the video is below the pre-determined threshold, determining if the fluid pump is operating at a maximum allowable pressure setting.

Optionally, if it is determined that the fluid pump is not operating at the maximum allowable pressure setting, increasing a pressure setting of the fluid pump.

Optionally, wherein if it is determined that the clarity of the video is above the pre-determined threshold, determining if the fluid pump is operating above a minimum allowable pressure setting.

Optionally, if it is determined that the fluid pump is operating above the minimum allowable pressure setting, decreasing a pressure setting of the fluid pump.

Optionally, the fluid pump is for fluid inflow to the internal portion of the patient.

Optionally, wherein the fluid pump is for fluid outflow from the internal portion of the patient.

According to an aspect, a non-transitory computer readable storage medium storing one or more programs for controlling a fluid pump for use in surgical procedures, for execution by one or more processors of an electronic device that when executed by the device, cause the device to receive video data captured from an imaging tool configured to image an internal portion of a patient, detect disturbances within the received video data by identifying one or more visual characteristics in the received video, create a plurality of classification metrics for classifying disturbances in the video data, determine the presence of one or more conditions in the received video data based on the plurality of classification metrics and the one or more visual characteristics, and adjust the flow through or head pressure from the fluid pump based on the determined presence of the one or more conditions in the received video data.

Optionally, adjusting the flow through or head pressure from the fluid pump comprises adjusting one or more settings of the fluid pump.

Optionally, the device is further caused to capture one or more image frames from the received video data, and wherein detecting disturbances within the received video data comprises detecting disturbances within each captured image frame of the one or more image frames.

Optionally, detecting disturbances within the received video data comprises detecting an amount of blood in a frame of the received video.

Optionally, detecting an amount of blood in a frame of the received video includes identifying one or more bleed regions in the frame of the received video data, identifying a total imaged area in the frame of the received video data, calculating an area of each identified bleed region, calculating a ratio of a sum of the calculated areas of each identified bleed region over the total imaged area in the frame of the received video data, and comparing the calculated ratio with a pre-determined threshold.

Optionally, detecting an amount of blood in a frame of the received video comprises converting a color space of a frame of the received video data to a hue, saturation, value (HSV) color space.

Optionally, if the calculated ratio is greater than the pre-determined threshold, increasing a pressure setting of the fluid pump.

Optionally, detecting disturbances within the received video data comprises detecting the amount of debris in a frame of the received video.

Optionally, detecting the amount of debris in a frame of the received video includes identifying one or more pieces of debris in the frame of the received video data, determining the total number of pieces of debris identified in the received video data, and comparing the determined total number of pieces of debris identified in the received video data with a pre-determined threshold.

Optionally, identifying one or more pieces of debris in the frame of the received video data comprises applying a mean shift clustering process to the frame of the received video data and extracting one or more maximal regions generated by the means shift clustering process.

Optionally, detecting the amount of debris in a frame of the received video comprises converting a color space of a frame of the received video data to a hue, saturation, value (HSV) color space.

Optionally, if the determined total number of pieces of debris identified in the received video data is greater than the pre-determined threshold, increasing a pressure setting of the fluid pump.

Optionally, the one or more disturbance detection processes comprises detecting a snow globe effect in a frame of the received video.

Optionally, detecting a snow globe effect includes identifying one or more snowy area regions in the frame of the received video data, identifying a total imaged area in the frame of the received video data, calculating an area of each identified snowy area region, calculating a ratio of a sum of the calculated areas of each identified snowy area region over the total imaged area in the frame of the received video data, and comparing the calculated ratio with a pre-determined threshold.

Optionally, detecting a snow globe effect comprises converting a color space of a frame of the received video data to a hue, saturation, value (HSV) color space.

Optionally, if the calculated ratio is greater than the pre-determined threshold, increasing a pressure setting of the fluid pump.

Optionally, if the calculated ratio is greater than the pre-determined threshold, increasing the fluid suction from a shaver tool located in the internal portion of the patient.

Optionally, detecting disturbances within the received video data comprises detecting turbidity in a frame of the received video.

Optionally, detecting turbidity in a frame of the received video includes applying a Laplacian of Gaussian kernel process to the frame of the received video, calculating a blur score based on the application of the Laplacian of Gaussian kernel process to the frame of the received video, and comparing the calculated blur score with a pre-determined threshold.

Optionally, if the calculated blur score is greater than the pre-determined threshold, increasing a pressure setting of the fluid pump.

Optionally, detecting turbidity in a frame of the received video comprises converting a color space of a frame of the received video data to a gray color space.

Optionally, the fluid pump is for fluid inflow to the internal portion of the patient.

Optionally, the fluid pump is for fluid outflow from the internal portion of the patient.

### BRIEF DESCRIPTION OF THE FIGURES

The invention will now be described, by way of example only, with reference to the accompanying drawings, in which:

FIG. 1 illustrates an exemplary endoscopy system according to examples of the disclosure.

FIG. 2 illustrates an exemplary method for controlling a surgical pump according to examples of the disclosure.

FIG. 3 illustrates an exemplary image processing process flow according to examples of the disclosure.

FIG. 4 illustrates an exemplary method for annotating images according to examples of the disclosure.

FIG. 5 illustrates an exemplary default pressure initialization process according to examples of the disclosure.

FIG. 6 illustrates an exemplary instrument suction activation process according to examples of the disclosure.

FIG. 7 illustrates an exemplary image clarity based process for controlling a surgical pump according to examples of the disclosure.

FIG. 8 illustrates an exemplary process for detecting blood in an image according to examples of the disclosure.

FIG. 9 illustrates an exemplary endoscopic image with segmented bleed regions according to examples of the disclosure.

FIG. 10 illustrates an exemplary process for detecting debris in an image according to examples of the disclosure.

FIG. 11 illustrates an exemplary endoscopic image with identified debris clusters according to examples of the disclosure.

FIG. 12 illustrates an exemplary process for detecting a snow globe effect in an image according to examples of the disclosure.

FIG. 13 illustrates an exemplary endoscopic image with segmented snowy area regions according to examples of the disclosure.

FIG. 14 illustrates an exemplary process for detecting turbidity in an image according to examples of the disclosure.

FIG. 15 illustrates an exemplary process for adjusting the settings of a surgical pump based on the image clarity according to examples of the disclosure.

FIG. 16 illustrates an exemplary computing system, according to examples of the disclosure.

### DETAILED DESCRIPTION

Reference will now be made in detail to implementations and examples of various aspects and variations of systems and methods described herein. Although several exemplary variations of the systems and methods are described herein, other variations of the systems and methods may include aspects of the systems and methods described herein combined in any suitable manner having combinations of all or some of the aspects described.

Described herein are systems and methods for automatically controlling a surgical pump for purposes of regulating fluid pressure in an internal area of a patient using video data taken from an endoscopic device. The endoscopic device may have been pre-inserted into the internal area prior to the start of the method. According to various examples, one or more images are captured from a video feed recorded from an endoscope during a surgical procedure. The captured images (i.e., images frames), in one or more examples, can be processed using one or more machine learning classifiers that are configured to determine the existence of various conditions that are occurring in the visualized internal area of a patient. For instance in one or more examples, the machine learning classifiers can be configured to determine the joint type depicted in the image, the instruments present in the image, the procedure step that the image depicts, as well as the presence/absence of visual disturbances present in the visualized internal portion of a patient. In addition to using machine learning classifiers, in one or more examples, the systems and methods described herein can also employ other processes for determining the presence of visual disturbances in a given image. For instance, and as described in further detail below, the images captured from the video data can be processed using one or more processes to determine the presence or absence of certain visual disturbances such as blood, debris, snow globe effects, turbidity, etc.

According to an aspect, the conditions determined by the one or more machine learning classifiers or processes can be used to determine an adjusted pressure setting of a surgical pump. The conditions determined by the one or more machine learning classifiers or processes can be used to control the pressure of the surgical pump. The method can exclude a step of providing an adjusted pressure by the pump. In one or more examples, the video data from the endoscopic imaging device can be used to determine a procedure step that is occurring in the image taken from the video data. Based on the determined procedure step, the default pressure setting pertaining to the determined procedure step can be retrieved and applied to the surgical pump so as to set the pressure inside the internal area to a pressure that is appropriate for the determined surgical step. In one or more examples, the pressure setting to be applied by the surgical pump can be set based on what instruments are determined to be present in the internal area as depicted in the images captured from the endoscopic video data. In one or more examples, the images can be processed by one or more machine learning classifiers to determine whether an instrument is found in the image. If an instrument is detected, further machine learning classifiers can be applied to the image to determine if the instrument is of the type that has its own dedicated suction (such as an RF probe or shaver). In one or more examples, the surgical pump can be made to work with the dedicated suction capabilities of the instruments found in an image so as to provide overall pressure management in the surgical space.

According to an aspect, the pressure to be applied by a surgical pump can be based on the determined presence or absence of visual disturbances detected in the image. In one or more examples, one or more image processing techniques can be applied to a captured image to determine the presence of such visual disturbances as blood, debris, snow globe effect, turbidity, etc. Based on the determined presence of these visual disturbances, the surgical pump can be controlled to increase pressure when these disturbances are detected or decreased when the disturbances are found to not be present.

In the following description of the various examples, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The present disclosure in some examples also relates to a device for performing the operations described herein. This device may be specially constructed for the required purposes, or it may comprise a general purpose computer selectively activated or reconfigured by a computer program stored in the computer. Such a computer program may be stored in a non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each connected to a computer system bus. Furthermore, the computing systems referred to in the specification may include a single processor or may be architectures employing multiple processor designs, such as for performing different functions or for increased computing capability. Suitable processors include central processing units (CPUs), graphical processing units (GPUs), field programmable gate arrays (FPGAs), and ASICs. In one or more examples, the systems and methods presented herein, including the computing systems referred to in the specification may be implemented on a cloud computing and cloud storage platform.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the present disclosure as described herein.

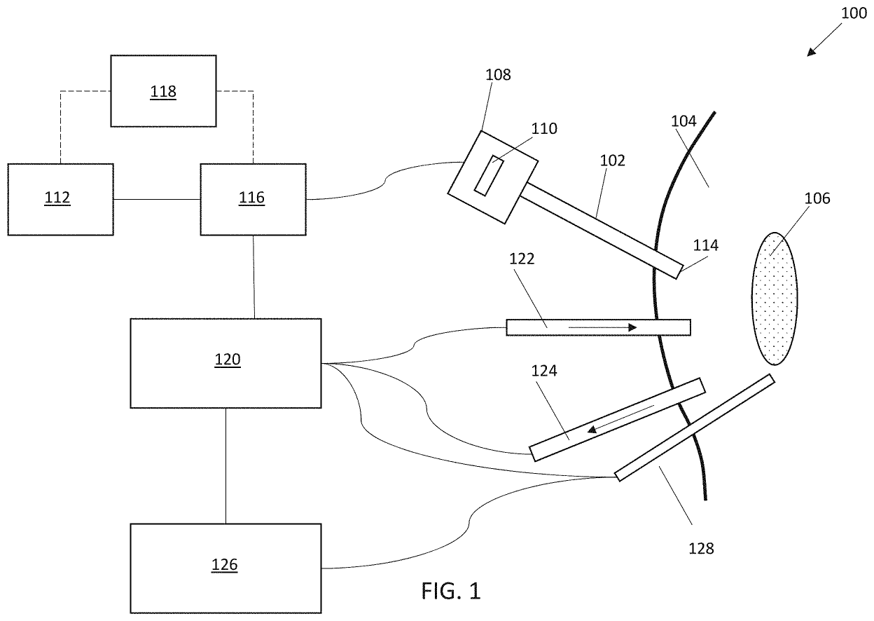

FIG. 1 illustrates an exemplary endoscopy system according to examples of the disclosure. System 100 includes an endoscope 102 for insertion into a surgical cavity 104 for imaging tissue 106 within the surgical cavity 104 during a medical procedure. The endoscope 102 may extend from an endoscopic camera head 108 that includes one or more imaging sensors 110. Light reflected and/or emitted (such as fluorescence light emitted by fluorescing targets that are excited by fluorescence excitation illumination light) from the tissue 106 is received by the distal end 114 of the endoscope 102. The light is propagated by the endoscope 102, such as via one or more optical components (for example, one or more lenses, prisms, light pipes, or other optical components), to the camera head 108, where it is directed onto the one or more imaging sensors 110. In one or more examples, one or more filters (not shown) may be included in the endoscope 102 and/or camera head 108 for filtering a portion of the light received from the tissue 106 (such as fluorescence excitation light). While the example above describes an example implementation of an imaging device, the example should not be seen as limiting to the disclosure and the systems and methods described herein can be implemented using other imaging devices that are configured to image the internal area of a patient.

The one or more imaging sensors 110 generate pixel data that can be transmitted to a camera control unit 112 that is communicatively connected to the camera head 108. The camera control unit 112 generates a video feed from the pixel data that shows the tissue being viewed by the camera at any given moment in time. In one or more examples, the video feed can be transmitted to an image processing unit 116 for further image processing, storage, display, and/or routing to an external device (not shown). The images can be transmitted to one or more displays 118, from the camera control unit 112 and/or the image processing unit 116, for visualization by medical personnel, such as by a surgeon for visualizing the surgical field 104 during a surgical procedure on a patient.

The imaging processing unit 116 can be communicatively coupled to an endoscopic surgical pump 120 configured to control the inflow and outflow of fluid in an internal portion of a patient. As described in further detail below, the imaging processing unit 116 can use the video data it processes to determine an adjusted pressure setting for the surgical pump 120, usable for regulating the pressure at an internal area of a patient such as surgical cavity 104. The imaging processing unit 116 can use the video data it processes to control the surgical pump 120 so as to regulate the pressure at an internal area of a patient such as surgical cavity 104. The surgical pump 120 can include an inflow portion 122 configured to deliver a clear fluid such as saline into the surgical cavity 104. The surgical pump 120 can also include a dedicated suction portion 124 configured to suction fluid out of the surgical cavity 104. In one or more examples, the surgical pump 120 is configured to regulate the internal pressure of the surgical cavity by either increasing or decreasing the rate at which the inflow portion 122 pumps fluid into the surgical cavity 104 or by increasing/decreasing the amount of suction at suction portion 124. In one or more examples, the surgical pump can also include a pressure sensor that is configured to sense the pressure inside of surgical cavity 104 during a surgical procedure.

In one or more examples, the system 100 can also include a tool controller 126 that is configured to control and/or operate a tool 128 used in performing a minimally invasive surgical procedure in the surgical cavity 104. In one or more examples, the tool controller (or even the tool itself) is communicatively coupled to the surgical pump 120. As will be described in further detail below, the tool 128 may include a suction component that can also work to suction out fluids and debris from the surgical cavity 104. By communicatively coupling the tool 128 and the surgical pump 124, the surgical pump can coordinate the actions of its own dedicated suction component 124 as well as the suction component of the tool 128 to regulate the pressure of the surgical cavity 104 as will be further described below. In one or more examples, and as illustrated in FIG. 1, the dedicated suction component of tool 128 can be controlled specifically by a suction pump that is a part of surgical pump 124.

As described above, different scenarios and conditions taking place inside of surgical cavity 104 can require that the inflow or outflow (or both) of surgical pump 120 be adjusted. For instance, different procedure steps during a surgical procedure may have different pressure needs. Furthermore, visibility conditions within a surgical cavity may require an increase or decrease in the inflow and outflow of surgical pump 120. For instance, an increase of blood within the surgical cavity 104 can require that the rate of inflow (which subsequently increases the pressure in the surgical cavity 104) be increased so as to arrest or minimize the bleeding. Conventionally, a surgeon would need to recognize a need to increase or decrease the pressure and then manually adjust the setting on the surgical pump to obtain the desired pressure. This process can interrupt the surgical procedure itself as the surgeon would need to stop with the procedure to make the necessary adjustments to the surgical pump 120, and further requires that the surgeon constantly assess whether the current pressure in the surgical cavity 104 is correct for the given conditions of the surgery.

Automating the process of detecting conditions associated with changing surgical pump pressure, as well as the process of adjusting the pressure setting for the surgical pump can thus reduce the cognitive load of the surgeon performing a surgery, but in one or more examples can also ensure that the pressure inside a surgical is controlled with precision. In this way, the surgical pump can provide a sufficient amount of pressure needed to manage the surgical cavity (i.e., provide good visualization), while at the same time ensuring that the pressure isn't so great as to cause injury or damage to the patient (i.e., by causing minimal extravasation).

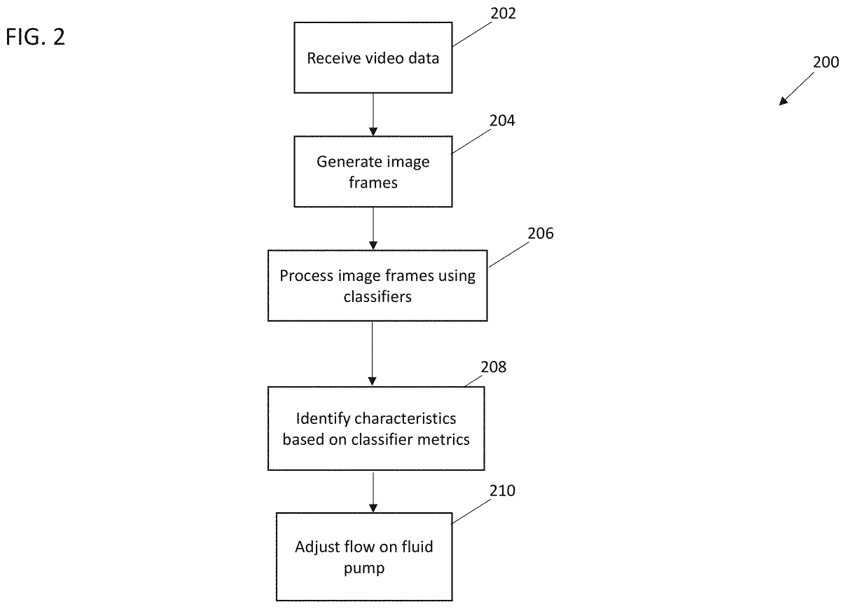

FIG. 2 illustrates an exemplary method for controlling a surgical pump according to examples of the disclosure. In one or more examples of the disclosure, the process 200 illustrated in FIG. 2 can begin at step 202 wherein video data from an endoscopic device or other type of imaging device is received. In one or more examples, the video data can be transmitted to one or more processors configured to implement process 200 using a High-Definition Multimedia Interface (HDMI), Digital Visual Interface (DVI) or other interface capable of connecting a video source (such as an endoscopic camera) to a display device or graphics processor.

Once the video data has been received at step 202, the process 200 can move to step 204 wherein one or more image frames can be extracted from the video data. In one or more examples, the image frames can be extracted from the video data in a periodic interval at a pre-determined period. Alternatively or additionally, one or more image frames can be extracted from the video data in response to user input such as for instance the surgeon pushing a button or other user input device to indicate that they want to capture an image from the video data at any particular moment in time. In one or more examples, the images can be extracted and stored in memory according to known image storage standards for memory such as JPEG, GIF, PNG, and TIFF image file formats. In one or more examples, the pre-determined time between capturing image frames from the video data can be configured to ensure that an image is captured during each stage in surgical procedure, thereby ensuring that the captured images will adequately represent all of the steps in a surgical process. In one or more examples, the image frames can be captured from the video data in real-time, i.e., as the surgical process is being performed. In one or more examples, and as part of step 204, the captured images can be reduced in size and cropped so as to reduce the amount of memory required to store a captured image. In one or more examples, the process of generating image frames from the received video data can be optional and the process 200 of FIG. 2 can be directly executed upon the video data from the endoscopic imaging device itself without requiring the capture of images from the video feed.

Once the image frames have been captured in step 204, the process 200 can move to step 206 wherein the image frames are processed using one or more classifiers that are configured to determine whether the captured image includes one or more characteristics. In one or more examples of the disclosure, the classifiers can include machine learning classifiers that are trained using a supervised learning process to automatically detect various features and characteristics contained within a given image or video feed. In one or more examples, and as described further in detail below, the one or more classifiers can include one or more image processing algorithms that are configured to identify various features and characteristics contained within a given image or video or feed. In one or more examples of the disclosure, the one or more classifiers of step 206 can include a combination of both machine learning classifiers and image processing algorithms that are collectively configured to determine one or more features or characteristics of the images associated with the pressure provided by a surgical pump during a minimally invasive surgery.

The one or more machine classifiers can be configured to identify the anatomy that is being shown in a given image. For instance, and as discussed in further detail below, the one or more machine classifiers can be configured to identify a particular joint type shown in an image such as whether a given image is of a hip, a shoulder, a knee, or any other anatomical feature that can be viewed using an imaging tool such as an endoscope. In one or more examples, and as further discussed in detail below, the one or more machine classifiers can be created using a supervised training process in which one or more training images (i.e., images that are known to contain specific anatomical features) can be used to create a classifier that can determine if an image inputted into the machine classifier contains a particular anatomical feature. Alternatively or additionally, the one or more machine learning classifiers can be configured to determine a particular surgical step being performed in the image. For instance, and as an example, the one or more machine classifiers can be configured to determine if a particular image shows a damaged anatomy (i.e., before the surgical procedure has taken place) or if the image shows the anatomy post repair.

Multiple machine classifiers can be configured to work collectively with one another to determine what features are present in a given image. As an example, a first machine learning classifier can be used to determine if a particular anatomical feature is present in a given image. If the machine classifier finds that it is more likely than not that the image contains a particular anatomical feature, then the image can be sent to a corresponding machine learning classifier to determine what procedure step is shown in the image. For instance if it is determined that a particular image shows a hip joint, then that same image can also be sent to a machine learning classifier configured to determine if the image shows a torn labrum as well as a separate machine learning classifier configured to determine if the image shows a labrum post-repair. However, if the machine learning classifier configured to determine if a given image shows a hip joint determines that it is unlikely that the image shows a hip joint, then the process 200 at step 206 may not send that image to a machine classifier corresponding to a procedure step for a surgery involving a hip (i.e., a torn labrum or a repaired labrum).

The one or more machine classifiers can include one or more image clarity classifiers that are configured to determine how clear or obscure a particular image is. During a surgical procedure certain conditions can obfuscate or make an image unclear. For instance the presence of blood, turbidity, bubbles, smoke, or other debris in a given image can indicate a need to increase the inflow of fluid from the surgical pump so as to remove the visual impairments from the surgical cavity.

The one or more machine classifiers are configured to generate a classification metric that is indicative of whether or not a particular feature (that the machine classifier is configured to determine) exists within a particular image. Thus, rather than making a binary determination (yes or no) as to whether a particular image includes a particular image, the classification metric can inform the process as to how likely it is that a particular image includes a particular feature. As an example, a machine classifier that is configured to classify whether an image contains a hip joint can output a classification metric in the range of 0 to 1 with 0 indicating that it is extremely unlikely that a particular image shows a hip joint and 1 indicating that it is extremely likely that a particular image shows a hip joint. Intermediate values between 0 and 1 can indicate the likelihood that an image contains a particular feature. For instance if a machine learning classifier outputs a 0.8, it can mean that it is more likely than not that the image shows a hip joint, while a classification metric of 0.1 means that it is not likely that the image contains a hip joint.

The one or more machine classifiers can be implemented using one or more convolutional neural networks (CNNs). CNNs are a class of deep neural networks that can be especially used to analyzing visual imagery to determine whether certain features exist in an image. Each CNN used to generate a machine classifier used at step 306 can include one or more layers, with each layer of the CNN configured to aide in the process of determining whether a particular image includes a feature that the overall CNN is configured to determine. Alternatively or additionally, the CNNs can be configured as Region-based Convolutional Neural Networks (R-CNNs) that can not only determine if a particular image contains a feature, but can identify the specific location in the image where the feature is shown.

Returning to the example of FIG. 2, once the one or more images have been processed by the one or more classifiers at step 206, the process 200 can move to step 208 wherein a determination is made as to what features are present within a particular image. The determination made at step 208 can be based on the classification metrics output from each of the classifiers. As an example, each of the classification metrics generated by each of the classifiers can be compared to one or more pre-determined thresholds, and if the classification metrics exceeds the pre-determined threshold than a determination is made that the image contains the feature corresponding to that machine learning classifier. As an example, if a machine learning classifier processing an image outputs a classification metric of 0.7, and the pre-determined threshold is set at 0.5, then at step 208 a determination is made that the image shows the feature associated with the classifier. In one or more examples, a determination can be made for each and every classifier that the image is processed through.

Once the characteristics of a given image, set of images, or the video feed have been determined at step 208, the process 200 can move to step 210 wherein an adjusted flow setting for flow through the surgical pump is determined based on the determined presence of one or more characteristics. The step 210 can include adjusting the flow through the surgical pump based on the predetermined presence of one or more characteristics. Adjusting the flow can include decreasing or increasing the flow rate of fluid pumped into a surgical cavity by the surgical pump. In one or more examples, step 210 can additionally or alternative include determining an adjusted the overall pressure setting for the pump. The step 210 can include adjusting the overall pressure provided by the pump to the surgical cavity. In one or more examples of the disclosure, the surgical pump can be implemented as a peristaltic pump that controls the joint pressure through increasing and decreasing the inflow rate. Alternatively or additionally, the surgical pump can be implemented as a propeller that generates a head pressure, which can be used to drive the pressure in a joint or surgical cavity. Thus, in one or more examples, adjusting the pump at step 210 can include adjusting both a flow driven pump or a pressure driven pump as described above.

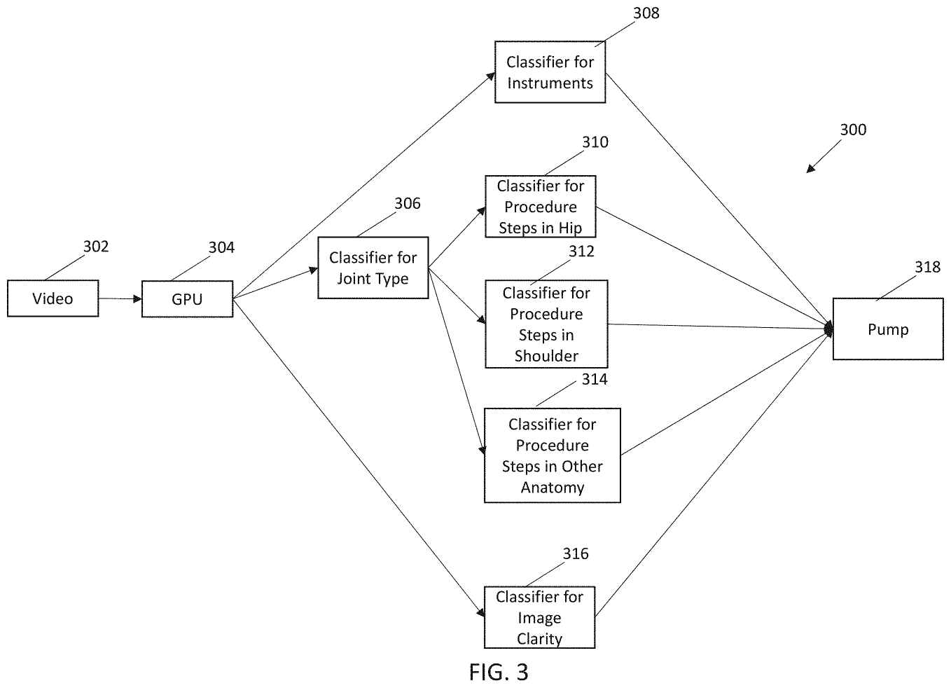

FIG. 3 illustrates an exemplary image processing process flow according to examples of the disclosure. In one or more examples, the process flow 300 illustrates an example implementation of the process described above with respect to FIG. 2. In one or more examples, the process can begin with the video data being received as described above at step 202 with respect to FIG. 2. In one or more examples, the video data can be transmitted to a graphics processing unit (GPU) 304, wherein the one or more image frames are generated from the video data as described above with respect to step 204 of FIG. 2.

Once the image frames have been generated at the GPU at 304, the classifiers can be applied to the images so as to ultimately determine what conditions (if any) are present in a given image or video that may require adjustment to the flow settings or pressure settings of the surgical pump. As shown in FIG. 3, in one or more examples, a given image can be sent to one or more classifiers 306 that are configured to determine the joint type shown in the image. In one or more examples, classifier 306 can be implemented as one or more separate machine learning classifiers configured to determine a joint type shown in the image or video. In one or more examples, once the image is processed using the one or more machine learning classifiers for joint type at 306, the image can be processed by one or more classifiers configured to determine the procedure step shown in the image. For instance, if it is determined that the image shows a hip joint (or is likely to show a hip joint) then the image can be sent to a classifier that is specifically configured to determine a procedure step for procedures that occur in a hip joint as depicted at 310. If however, the image is determined to be of a shoulder joint, then the image can be sent to one or more classifiers configured to determine a procedure step for the shoulder as depicted at 310. Similarly, and as depicted at 314, the image can be sent to one or more machine classifiers configured to determine procedure steps in other anatomical features of the body as depicted at 314. In one or more examples of the disclosure, other anatomical features can also include determining when the endoscopic device is not inserted into the patient (i.e., no anatomy shown) in which case, the inflow of the pump can be turned off at 318.

As will be described in further detail below, determining the anatomy and procedure step from a given surgical image or video feed can be used to determine a pressure or flow setting of the surgical pump. In one or more examples, the one or more classifiers for instruments 318 can be implemented as one or more machine learning classifiers implemented using a supervised training process.

In addition to determining the joint type and procedure step, in one or more examples, the GPU 304 can transmit image or video data to one or more classifiers configured to determine the presence of an instrument in a given image or video as depicted at 308. As will be described in detail further below, certain surgical instruments can include their own suction capabilities, which can influence the inflow and outflow rates of a surgical pump. Thus, in one or more examples, the one or more classifiers can include one or more classifiers configured to determine the presence (or absence) of various instruments in the surgical cavity as depicted at 308. In one or more examples, the classifier for instruments 308 can include multiple classifiers, each classifier configured to determine the presence of a single instrument. For instance, classifiers 308 can include a classifier configured to determine if a shaver is in the surgical cavity and if that shaver is a cutter or bur. A separate classifier for determining if an RF probe is found in the surgical cavity (via the images or video taken by an endoscopic imaging device in the surgical cavity) can be configured. In one or more examples, the one or more classifiers for instruments 308 can be implemented as one or more machine learning classifiers implemented using a supervised training process.

The one or more classifiers can be configured to determine various conditions associated with the clarity of an image as depicted at 316. As described above, and described in detail below, various conditions that can inhibit the clarity of a video such as blood, debris, snow globe conditions, and turbidity, if detected, can require a change to the pressure and/or flow settings of the surgical pump. Additionally or alternatively, the image clarity classifiers can also be configured to detect when an internal portion of a patient has collapsed due to lack of pressure. Thus, in one or more examples, the one or more machine classifiers can be configured to determine these conditions. In one or more examples, each condition relating to clarity can be implemented as its own classifier (for efficiency each of these classifiers are depicted by a single block at 316). In one or more examples, the one or more classifiers for image clarity 316 can be implemented as one or more machine learning classifiers implemented using a supervised training process. Alternatively or additionally, the one or more classifiers for image clarity 316 can be implemented using one or more image processing algorithms configured to determine the presence of any of the one or more image clarity conditions described above.

As described above with respect to FIG. 2, the outputs of each classifier depicted in the system 300 can be transmitted to the surgical pump to determine if any adjustments to the inflow/outflow or pressure of the pump are necessary in light of the characteristics determine at least in part by the one or more classifiers described above with respect to FIG. 3. As described above, adjustments to the pump can include increasing or decreasing the inflow of fluid provided by the pump to a surgical cavity, and in one or more examples, can also include increasing or decreasing the outflow of the surgical pump by for instance increasing or decreasing the rate of suction of the pump. In one or more examples, the pump as depicted at 318 can input the determinations from each of the classifiers in the system 300 and make a determination as to the necessary adjustments to the inflow, outflow, or pressure needed in response to the determined conditions based on the output of each classifier depicted in FIG. 3. In this way, the surgical pump can make decisions as to pressure needs at any given moment during a surgery based on a plurality of conditions that may occur during a surgery as described in further detail below.

As described above, each of the classifiers depicted in FIG. 3 can be implemented as machine learning classifiers that are generated using a supervised training process. In a supervised training process, the classifier can be generated by using one or more training images. Each training image can be annotated (i.e., by appending metadata to the image) that identifies one or more characteristics of the image. For instance, using a hip joint machine learning classifier configured to identify the presence of a hip joint in an image as an example, the machine learning classifier can be generated using a plurality of training images known (a priori) to visualize hip joints.

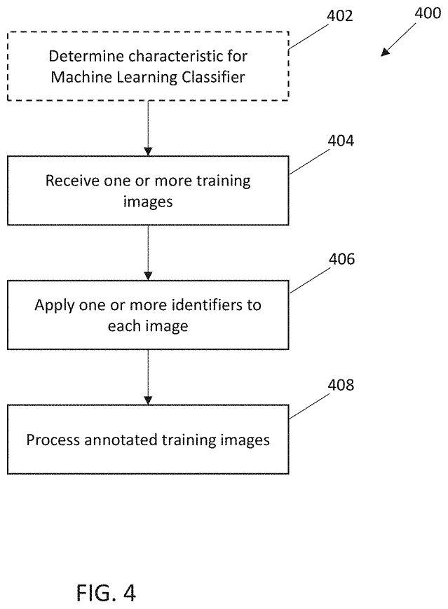

FIG. 4 illustrates an exemplary method for annotating images according to examples of the disclosure. In the example of FIG. 4, the process 400 can begin at step 402 wherein a particular characteristic for a given machine learning classifier is selected or determined. In one or more examples, the characteristics can be selected based on the conditions that can influence the inflow, outflow, and/or pressure requirements of a surgical pump during a surgical procedure. Thus, for instance, if a particular medical practice only performs procedures involving hip joints, then the characteristics determined or selected at step 402 will include only characteristics germane to hip surgery contexts. In one or more examples, step 402 can be optional, as the selection of characteristics needed to for the machine learning classifiers can be selected beforehand in a separate process.

Once the one or more characteristics to be classified have been determined at step 402, the process 400 can move to step 404 wherein one or more training images corresponding to the selected characteristics are received. In one or more examples, each training image can include one or more identifiers that identify the characteristics contained within an image. The identifiers can take the form of annotations that are appended to the metadata of the image, identifying what characteristics are contained within the image. A particular image of the training image set can include multiple identifiers. For instance a picture of a repaired labrum tear can include a first identifier that indicates the picture contains a hip joint and a separate identifier that indicates the procedure step, which in the example is a repaired labrum.

If the training images received at step 404 do not include identifiers, then the process can move to step 406 wherein one or more identifiers are applied to each image of the one or more training images. In one or more examples, the training images can be annotated with identifiers using a variety of methods. For instance, in one or more examples, the training images can be manually applied by a human or humans who view each training image, determine what characteristics are contained within the image, and then annotate the image with the identifiers pertaining to those characteristics. Alternatively or additionally, the training images can be harvested from images that have been previously classified by a machine classifier. For instance, and returning to the examples of FIG. 2, once a machine learning classifier makes a determination as to the characteristics contained within an image at step 208, the image can be annotated with the identified characteristics (i.e., annotated with one or more identifiers) and the image can then be transmitted to and stored in a memory for later use as a training image. In this way, each of the machine learning classifiers can be constantly improved with new training data (i.e., by taking information from previously classified images) so as to improve the overall accuracy of the machine learning classifier.

In one or more examples, and in the case of segmentation or region based classifiers such as R-CNNS, the training images can be annotated on a pixel-by-pixel or regional basis to identify the specific pixels or regions of an image that contain specific characteristics. For instance in the case of R-CNNs, the annotations can take the form of bounding boxes or segmentations of the training images. Once each training image has one or more identifiers annotated to the image at step 406, the process 400 can move to step 408 wherein the one or more training images are processed by each of the machine learning classifiers in order to train the classifier. In one or more examples, and in the case of CNNs, processing the training images can include building the individual layers of the CNN.

As described above, the particular anatomy and procedure step occurring during a surgical procedure can have an effect on the amount of pressure, inflow and/or outflow to be delivered by the surgical pump. For instance, a surgery in a knee may have different pressure requirements than a surgery taking place in an elbow. In addition to the anatomy, the procedure step happening at any given moment in time during a surgical procedure can also influence the pressure needs to be met by the surgical pump. For instance, in the beginning stages of a surgical procedure when there may still be damage in the anatomy being operated on, the surgical pump may be required to deliver higher pressure to a surgical cavity than if the surgery is in the stage when the anatomy has already been repaired. Furthermore, keeping increased pressure throughout a surgery may cause injury or damage to the patient, and thus as the surgery progresses, the surgical pump may be required to decrease the overall pressure in a surgical cavity. Thus, and as described in further detail below, the surgical pump can be configured to maintain a library of default pressure settings corresponding to the anatomy and procedure step determined by the one or more machine classifiers that are used to determine the anatomy and procedure step occurring at a given moment in time during a surgical procedure.

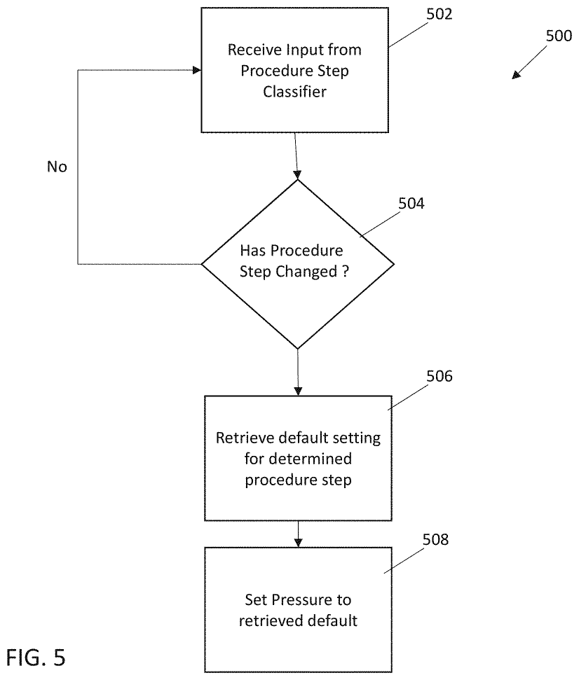

FIG. 5 illustrates an exemplary default pressure initialization process according to examples of the disclosure. The example of FIG. 5 illustrates an exemplary process for adjusting the pressure/flow settings of the surgical pump based on the identified anatomy and surgical procedure step determined to be present in a given one or more images or video taken from an endoscopic imaging device during a surgical procedure. In one or more examples of the disclosure, the process 500 depicted in FIG. 5 can begin at step 502 wherein data outputted by one or more classifiers associated with the anatomy and procedure step of a surgical procedure described above is received by a processor communicatively coupled to the surgical pump and configured to adjust the flow/pressure settings of the pump. Once the inputs from the classifiers are received at step 502, the process 500 can move to step 504 wherein a determination is made as to whether the procedure step has changed. In one or more examples, if it is determined at step 504 that the procedure step has not changed then the pressure settings of the surgical pump may not need to be adjusted and the process 500 can revert back to step 502 to receive further data from the one or more procedure step classifiers.

If however, a determination is made at step 504 that the procedure step has changed, the process 500 can move to step 506 wherein one or more default settings associated with the determined procedure step can be retrieved. As described above, each procedure step associated with a surgical procedure can have a default pressure setting associated with it. The default pressure setting can indicate the inflow/outflow or pressure that the pump should be set to when a particular procedure step in a given surgical procedure is being performed. As the surgery progresses and the procedure step changes, the default settings for the pump can change to account for the varying pressure needs at a given procedure step. Thus, at step 506, in light of a determination that the procedure step has changed, the default pressure setting associated with that particular procedure step can be retrieved and applied (in a subsequent step of process 500) to the surgical pump to adjust the pressure setting to a level commensurate with the requirements of that particular procedure step.

In one or more examples, once the default setting for the identified procedure step is retrieved at step 506, the process 500 can move to step 508 wherein the pressure setting associated with the retrieved default setting is applied to the surgical pump. In this way, the pressure setting of the surgical pump can be automatically adjusted as the surgery progresses rather than requiring the surgeon to manually adjust the pressure settings as the surgery progresses, thus reducing the manual and cognitive load placed on the surgeon while performing a surgical procedure.

As discussed above with respect to FIG. 3, in one or more examples, the required pressure settings of the surgical pump can be dependent on the instruments that are present and being used in a surgical cavity during a surgical procedure. Specifically, in one or more examples, one or more types of instruments used during a surgical procedure can also include its own suction. Because these types of instruments come with their own suction, the surgical pump can be required to adjust its inflow/outflow or pressure settings to account for the suction produced by other instruments. Conventionally, the surgeon recognizing that they are working with one or more surgical instruments that includes its own suction, would manually turn off the dedicated suction of the surgical pump (while keeping the inflow settings the same). However, as described above, using one or more classifiers that can automatically detect the presence or removal of instruments in the surgical cavity, the surgical pump can automatically adjust its settings to account for other instruments.

In one or more examples, the instruments (such as an RF probe or shaver) that include their own suction can be communicatively coupled to the surgical pump or controller configured to control the surgical pump so that the controller/pump can directly control the suction of those devices. In this way, the surgical pump can coordinate the actions of all of the devices that can contribute to the overall pressure in the joint so as to ensure that the pressure is comprehensively managed without intervention from the surgeon.

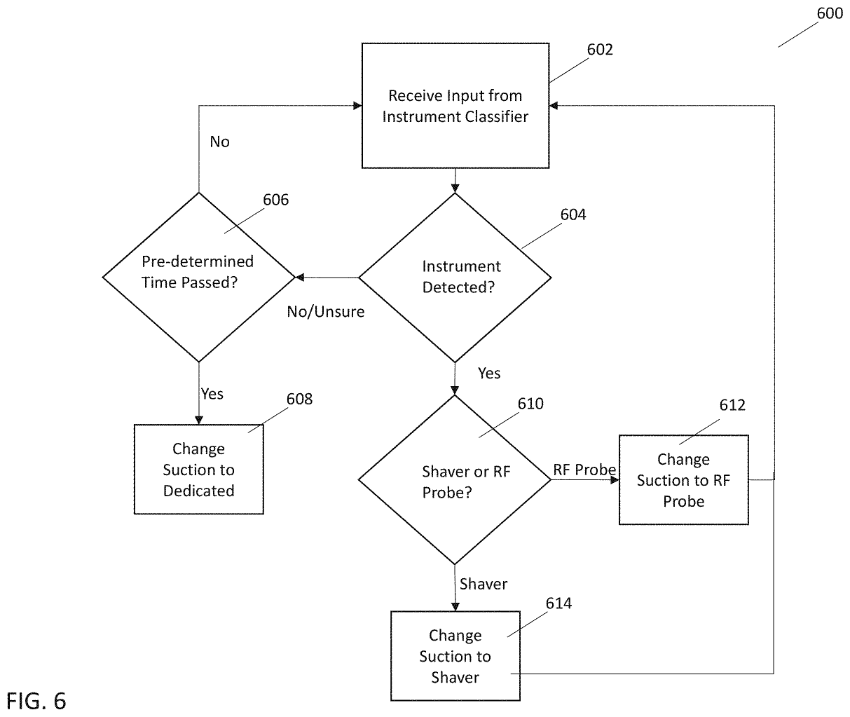

FIG. 6 illustrates an exemplary instrument suction activation process according to examples of the disclosure. The example of FIG. 6 illustrates an exemplary process for adjusting the pressure/flow settings of the surgical pump based on the instruments determined to be present in a given one or more images or video taken from an endoscopic imaging device during a surgical procedure. In one more examples of the disclosure, the process 600 depicted in FIG. 6 can begin at step 602 wherein data outputted by one or more classifiers configured to determine the type of instruments in a surgical cavity described above is received by a processor communicatively coupled to the surgical pump and configured to adjust the flow/pressure settings of the pump. Once the inputs from the classifiers are received at step 602, the process 600 can move to step 604 wherein a determination is made as to whether an instrument (associated with the one or more classifiers) is present in the images or video data of the endoscopic imaging device.

In one or more examples, if at step 604 it is determined that an instrument associated with the one or more instrument classifiers was detected then the process 600 can move to step 610 wherein a determination is made as to which device was detected based on the data from the one or more classifiers associated with instrument type. In the example of FIG. 6, the examples of shavers and RF probes are used for illustration, however the example should not be seen as limiting and can be applied to scenarios in which additional devices with their own suction are introduced into the surgical cavity. If it is determined at step 610 that an RF probe is present in the surgical cavity (based on the classifier data) then the process 600 can move to step 612 wherein the surgical pump (or a controller communicatively coupled to the surgical pump) can activate the RF probe's suction, and in one or more examples, deactivate the surgical pump's dedicated suction. Similarly, if it is determined at step 610 that a shaver is present in the surgical cavity then the process 600 can move to step 614 wherein the surgical pump/controller can activate the shaver's suction, and in one or more examples, deactivate the surgical pump's dedicated suction. In one or more examples, after both steps 612 and 614, the process 600 can revert back to step 602 so that the system can detect when the instrument has been removed (as to be further described below).

In one or more examples, if at step 604 it is determined that no instrument was detected or if the classifier is unsure that an instrument is in the surgical cavity (for instance if the classification metric is halfway between 0 and 1) the process 600 can move to step 606 wherein a determination is made as to whether a pre-determined time has passed since the classifiers began to not detect an instrument or were unsure that an instrument was present. In one or more examples, when an instrument is present in the surgical cavity but then “disappears” from the classifiers (i.e., the classifiers no longer see the instrument in the images), the disappearance may be caused by a momentary error in the classifier or because the instrument has been removed by the surgeon from the surgical cavity. If the disappearance is caused by a momentary error, reacting to that error by adjusting the surgical pump could propagate the error and cause an improper amount of pressure to be delivered via the surgical pump to the surgical cavity. Thus, in one or more examples, the process 600 can wait a pre-determined amount of time after an instrument disappears from the classifiers before adjusting the pressure or pressure setting to account for the removal of the instrument. At step 606, in one or more examples, the first time an instrument disappears from the classifiers, a timer can be started and the process can revert back to step 602 to receive additional data from the one or more instrument classifiers. Each time no instrument is detected at step 604, the process can go to step 606 to check if the pre-determined time has passed. If not, then the process again reverts back to step 602, thus creating a loop that is only broken if an instrument is detected in the surgical cavity, or the pre-determined time has passed since the disappearance of the instrument from the classifiers.

In one or more examples, once the pre-determined time has passed at step 600, the process 600 can move to step 608, wherein the surgical pump or controller that controls the surgical pump activates its own dedicated suction (i.e., suction 124) and in one or more examples, deactivates the suction of the instrument that was removed.

As described above with respect to FIG. 3, the system can include one or more image clarity classifiers. As described above, various conditions that can inhibit the clarity of a video such as blood, debris, snow globe conditions, and turbidity, if detected, can require a change to the pressure and/or flow settings of the surgical pump. Thus, in one or more examples, the one or more classifiers can be configured to determine these conditions. In one or more examples, and as described above, the one or more classifiers for image clarity 316 can be implemented as one or more machine learning classifiers implemented using a supervised training process. Alternatively or additionally, the one or more classifiers for image clarity 316 can be implemented using one or more image processing algorithms configured to determine the presence of any of the one or more image clarity conditions described above. In one or more examples, each clarity condition (i.e., blood, turbidity, snow globe, debris) can be implemented as its own classifier that applies an image processing algorithm that is configured to identify a particular visual disturbance that can affect the clarity of an image.

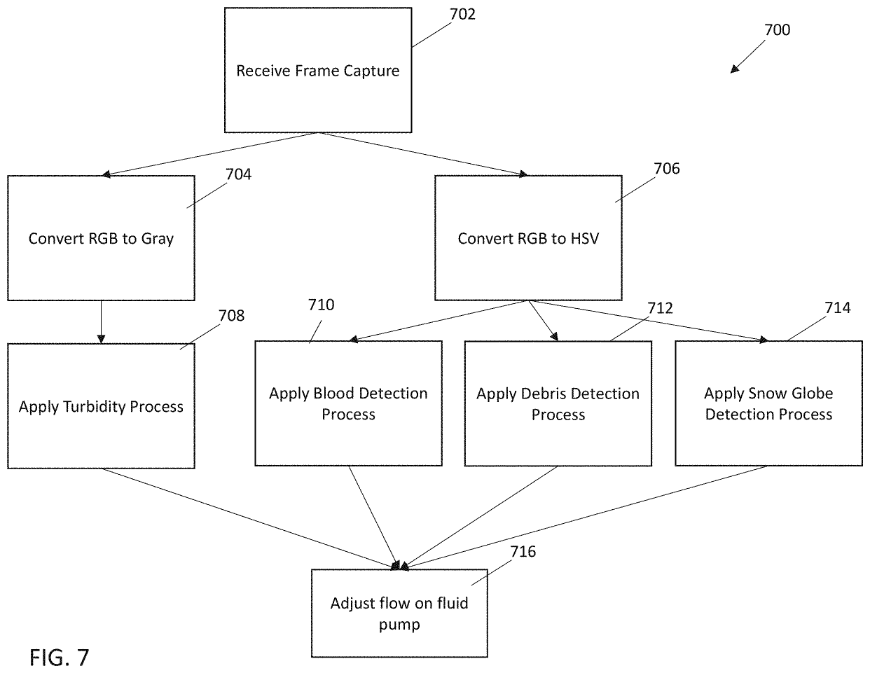

FIG. 7 illustrates an exemplary image clarity based process for controlling a surgical pump according to examples of the disclosure. The example of FIG. 7 illustrates a process 700 that takes as its input one or more images captured from an endoscopic imaging device video feed, and processes them to identify one or more types of visual disturbances present in the images, and uses the information to adjust the inflow/outflow or pressure settings of the surgical pump. In one or more examples, the process 700 can begin at step 702 wherein one or more captured image frames from an endoscopic imaging device video feed are received. Once the captured frames are received at step 702, each frame can be converted from a conventional red, green, blue (RGB) color space to one or more alternative color spaces that are configured to accentuate various visual phenomenon that can affect the clarity of a given image. Thus, in one or more examples, after receiving the captured image frames at step 702, the process 700 can simultaneously and in parallel convert a single image into two separate images with a modified color space as depicted at steps 704 and 706.

In one or more examples, at step 704, the one or more images received at step 702 can be converted from the RGB color space to the Grayscale color space. In the grayscale color space, each pixel rather than representing a particular color can instead represent an amount of light (i.e., an intensity). Converting an image from RGB to grayscale as described in further detail below can accentuate various features of the image that make it easier to identify certain visual phenomenon such as turbidity.

In one or more examples, at step 706, the one or more images received at step 702 can be converted from the RGB color space to the hue, saturation, value (HSV) color space. The HSV color space can describe colors in terms of their shade (i.e., amount of gray) and their brightness value). Converting an image from the RGB color space to the HSV color space can also be used to accentuate various features of the image that make it easier to identify certain visual phenomenon such as blood, debris, and a snow globe effect (described in further detail below). In one or more examples, after converting the one or more image from RGB to HSV at step 706, the process 700 can apply one or more image processing algorithms to the converted images to identify specific visual phenomenon (described in further detail below) as depicted in steps 710, 712, and 714.

In one or more examples, at step 710, the process 700 can apply a blood detection process to the converted image to detect the presence of blood in a given image. As described in further detail below, while some blood is to be expected during a surgical procedure, an excess amount of blood can create a visual impairment for the surgeon during a surgery and thus the surgical pump may need to be adjusted so as to apply more pressure in the surgical cavity so as to arrest or minimize the amount of blood present in the surgical cavity. In one or more examples, at step 712, the process 700 can apply a debris detection process to the converted image to detect the presence of debris in a given image. Debris can refer to particles in the surgical cavity that are unnecessary and can be caused by loose fibrous tissue or resected tissue/bone floating in the joint space fluid. In one or more examples, at step 714, the process 700 can apply a snow globe detection process to the converted image. In one or more examples, a “snow globe” effect can refer to debris generated by resecting bone that causes poor visibility in the joint space. Thus, at step 714, the snow globe detection process using the HSV color space image can perform an algorithm (described in further detail below) that can be used to identify a snow globe effect.

Referring back to step 704, the grayscale image can also be used to identify one or more visual phenomenon. For instance, in one or more examples of the disclosure, once an image has been converted from RGB to grayscale at step 704, the process 700 can move to step 708 wherein the grayscale image is used to determine the turbidity present in the image. In one or more examples, turbidity can refer to the cloudiness or haziness of a fluid caused by particles floating in a liquid medium. Thus, at step 708, an algorithm (described in detail below) can be applied to a grayscale image to determine turbidity levels in the image. Once each of the processes depicted at steps 708, 710, 712, and 714 have been performed, the process 700 can move to step 716 wherein the inflow, outflow, and/or pressure settings of the surgical pump can be adjusted based on the outcomes of the processes.

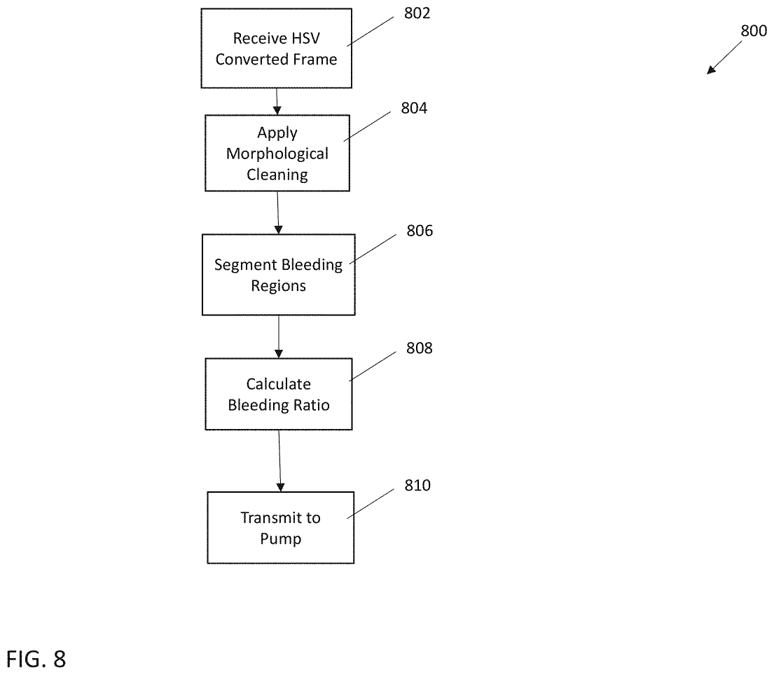

FIG. 8 illustrates an exemplary process for detecting blood in an image according to examples of the disclosure. In one or more examples, the process 800 can begin at step 802 wherein an HSV converted image frame (described above with respect to step 706 of FIG. 7) is received. In one or more examples, after the HSV converted image frame is received at step 802, the process 800 can move to step 804 wherein a morphological cleaning process is applied to the image. In one or more examples, a morphological cleaning process can refer to an image processing algorithm that can be applied to an image to grow or shrink image regions as well as remove or fill-in image region boundary pixels. The morphological cleaning process can be configured to enhance image regions (such as regions in which bleeding is present) so that they can be more easily identified.

After morphological cleaning is applied to the image at step 804, the process 800 can move to step 806 wherein one or more bleeding regions are segmented within the image. A “bleeding region” can refer to a region in the image in which blood is present. In one or more examples, a bleeding region can be identified based on the HSV characteristics of the pixels (i.e., pixels that contain HSV values that are indicative of blood). For instance, a bleeding or bleed region can be identified based on pixels that are within a certain range of HSV values. In one or more examples, segmenting the image can refer to identifying regions or segments in the image in which, based on the HSV values, blood is likely present. Once the bleeding regions have been segmented at step 806, the process 800 can move to step 808 wherein a ratio of the area covered by bleeding regions over the total area shown in the image is calculated. This ratio can represent how much blood is contained in a given image as a function of the percentage of space of the total image area occupied by bleeding regions. Thus, as an example, if a total image area is 100 pixels and the sum of all the bleeding regions occupies only 3 pixels then the ratio can be determined to be 3%, meaning that the bleeding regions occupy 3% of the total image area.

Once the ratio has been calculated at step 808, the process 800 can move to step 810 wherein the calculated ratio is transmitted to the pump or a controller communicatively coupled to the pump that can adjust the flow settings of the pump based on the determined ratio. The pre-determined threshold, in one or more examples, can be empirically determined. Additionally or alternatively, the pre-determined threshold can be set based on the surgeon's preferences. In one or more examples, the surgical pump can increase the pressure settings if the calculated ratio is greater than a pre-determined threshold. For instance, if the ratio is found to be 30% while the pre-determined threshold is 50% then the pump may take no action and leave the pressure settings of the pump as is. However, if during the surgery the ratio increases to 60%, then the pump may increase the pressure in an attempt to minimize or stop the bleeding in the surgical cavity. In one or more examples, the pump or a controller communicatively coupled to the pump can increase the pressure in a time-based manner. For example, if the determined ratio meets or exceeds the pre-determined threshold, a timer can be initiated to control the rate of increasing the pressure in the joint. In one or more examples the rate of increase can be based on the period of time that a visual disturbance is detected. For instance, the longer blood is detected in the joint, the faster the pressure increases (i.e., the rate increases). In one or more examples, the rate of increase can reset to zero when it is determined that there is no longer a visual disturbance, or only a minimal amount of visual disturbance.

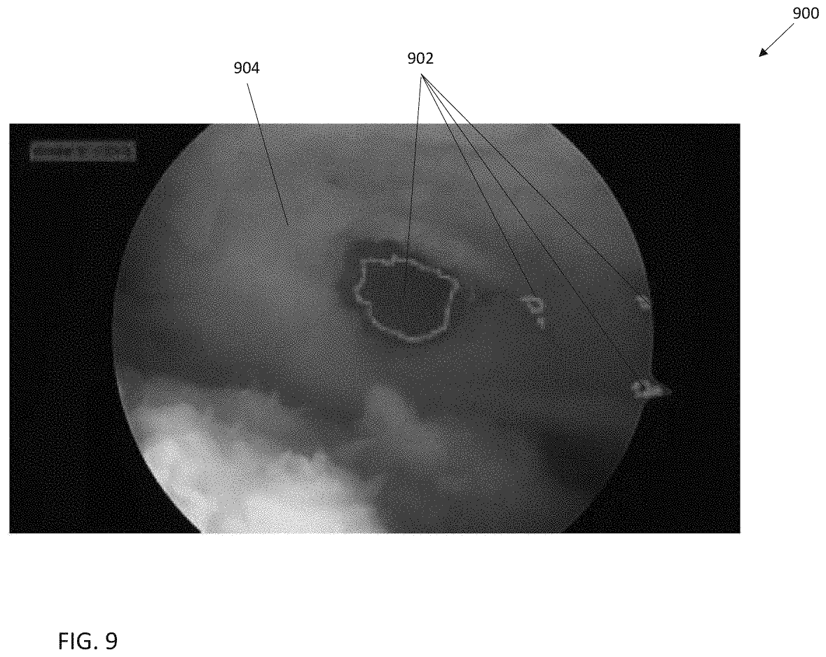

FIG. 9 illustrates an exemplary endoscopic image with segmented bleed regions according to examples of the disclosure. In the example of FIG. 9, the image 900 can include one or more bleed regions 902 as identified at step 806 in the example of FIG. 8. The example of FIG. 9 shows an image that contains a 3% bleed ratio, meaning that the identified bleed regions occupy about 3% of the total scope area.

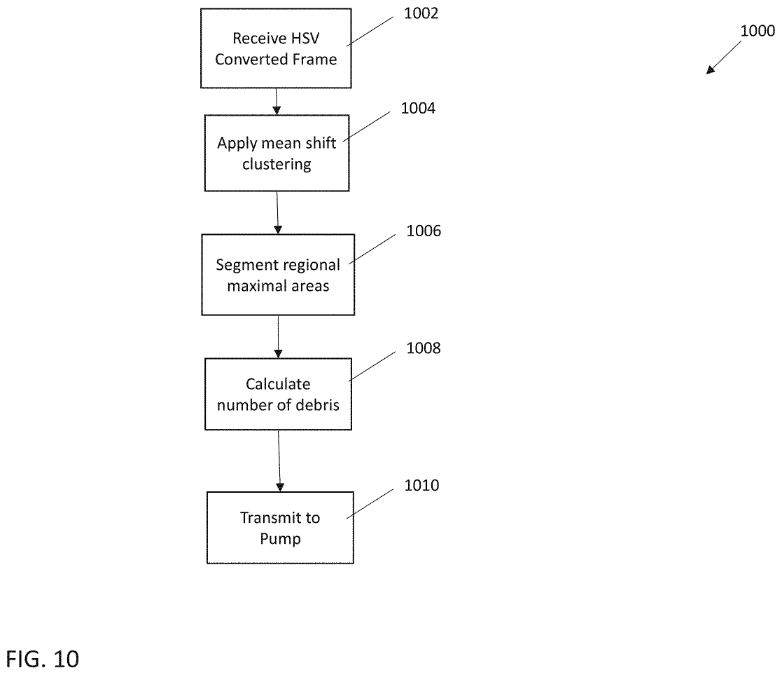

FIG. 10 illustrates an exemplary process for detecting debris in an image according to examples of the disclosure. In one or more examples, the process 1000 can begin at step 1002 wherein an HSV converted image frame (described above with respect to step 706 of FIG. 7) is received. With respect to debris, the HSV color space can make it easier to distinguish debris (i.e., loose fibrous tissue floating in the surgical space) from other tissue and objects that are imaged in a surgical cavity. As described above, this debris can represent visual impairments to a surgeon when performing a surgical procedure, and thus in order to automate the process of adjusting the pressure and/or outflow to remove or minimize debris the process should be able to automatically distinguish debris from other matter in the surgical cavity.

In one or more examples, after the HSV converted image frame is received at step 1002, the process 1000 can move to step 1004 wherein a mean shift clustering algorithm is applied to the received image frame. In one or more examples, the mean shift clustering algorithm can be configured to locate the local maxima of an image given data sampled from the image (i.e., the pixel values). In one or more examples, the debris in an image will appear as small areas in an image where the pixel values suddenly shift. The mean shift clustering algorithm can identify the areas in an image where the mean pixel values suddenly shift (i.e., local maxima) thus identifying individual pieces of debris in a given image.

Once the mean shift clustering algorithm is applied at step 1004, the process 1000 can move to step 1006 wherein the regional maximal areas/regions are segmented from the image. In one or more examples each regional maximal area can represent a piece of debris in the image. Thus by identifying these regions, and as described below, the process 1000 can calculate the specific number of debris pieces that are found within a given image. Once the regions have been segmented at step 1006, the process 1000 can move to step 1008 wherein the number of pieces of debris in a given image are counted. In one or more examples, counting pieces of debris can include simply counting the number of regional maximal areas identified in step 1006. Finally at step 1010, the number of debris can be transmitted to the surgical pump or a controller communicatively coupled to the pump so as to adjust the pressure settings of the pump based on the number of pieces of debris found in an image.

In one or more examples, the pump can be adjusted by increasing an amount of suction (i.e., outflow) that the pump is generating. By increasing the suction, the debris in a surgical cavity can be removed at a quicker rate to thereby remove the overall amount of debris in a surgical cavity and thereby removing or minimizing the visual impairments to the surgeon. In one or more examples, the amount of suction can be based on the number of pieces of debris found in the surgical cavity based on the images captured from the endoscopic imaging device. In one or more examples, the pump can also adjust the inflow of the fluid to sweep the debris out of the visualized area.

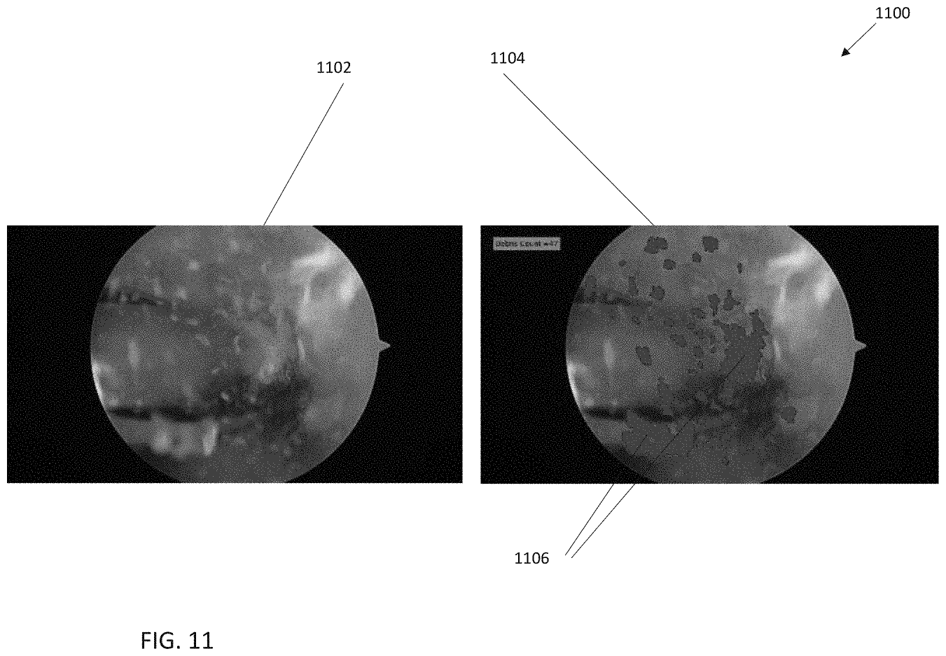

FIG. 11 illustrates an exemplary endoscopic image with identified debris clusters according to examples of the disclosure. The images 1100 of FIG. 11 can include a first image 1102 that illustrates an image with debris that has not been processed to identify the individual pieces of debris. Thus, the image 1102 illustrates an image with debris before the process described above with respect to FIG. 10 is applied to the image. The images 1100 include a second image 1104 that shows the identified debris pieces 1106 once the process described above with respect to FIG. 10 is applied to the image.

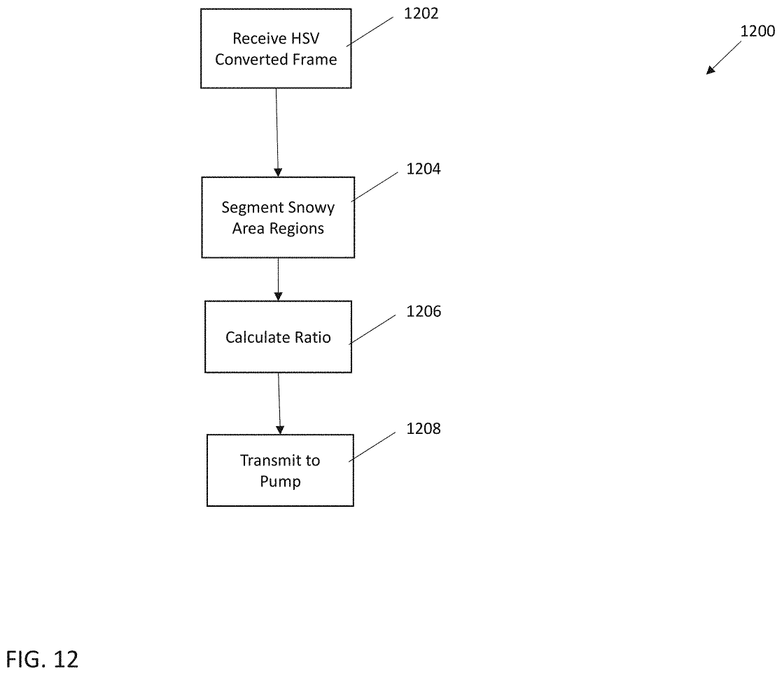

FIG. 12 illustrates an exemplary process for detecting a snow globe effect in an image according to examples of the disclosure. In one or more examples, the process 1200 can begin at step 1202 wherein an HSV converted image frame (described above with respect to step 706 of FIG. 7) is received. In one or more examples, after the HSV converted image frame is received at step 1202, the process 1200 can move to step 1204 wherein one or more snowy area regions are segmented within the image. A “snowy area region” can refer to a region in the image in which the snow globe effect (i.e., debris from resected bone) is present. In one or more examples, a snowy area region can be identified based on the HSV characteristics of the pixels (i.e., pixels that contain HSV values that are indicative of a snow globe effect). For instance, a snow globe region can be identified based on pixels that are within a certain range of HSV values. In one or more examples, segmenting the image can refer to identifying regions or segments in the image in which, based on the HSV values, the snow globe effect is likely present. Once the snowy area regions have been segmented at step 1204, the process 1200 can move to step 1206 wherein a ratio of the area covered by snowy area regions over the total area shown in the image is calculated. This ratio can represent how prevalent the snow globe effect is in a given image as a function of the percentage of space of the total image area occupied by snowy area regions. Thus, as an example, if a total image area is 100 pixels and the sum of all the snowy area regions occupies only 3 pixels then the ratio can be determined to be 3%, meaning that the snowy area regions occupy 3% of the total image area.

Once the ratio has been calculated at step 1206, the process 1200 can move to step 1208 wherein the calculated ratio is transmitted to the pump or a controller communicatively coupled to the pump that can determine an adjusted the flow settings of the pump based on the determined ratio. In one or more examples, the surgical pump can increase the pressure settings if the calculated ratio is greater than a pre-determined threshold. For instance, if the ratio is found to be 30% while the pre-determined threshold is 50% then the pump may take no action and leave the pressure settings of the pump as is. However, if during the surgery the ratio increases to 60%, then the pump may increase the pressure in an attempt to minimize or remove the debris from resected bone in the surgical cavity. The pre-determined threshold, in one or more examples, can be empirically determined. Additionally or alternatively, the pre-determined threshold can be set based on the surgeon's preferences. In one or more examples, rather than increasing the pressure, the pump can be adjusted to increase the suction so as to remove the resected bone that is causing the snow globe effect.

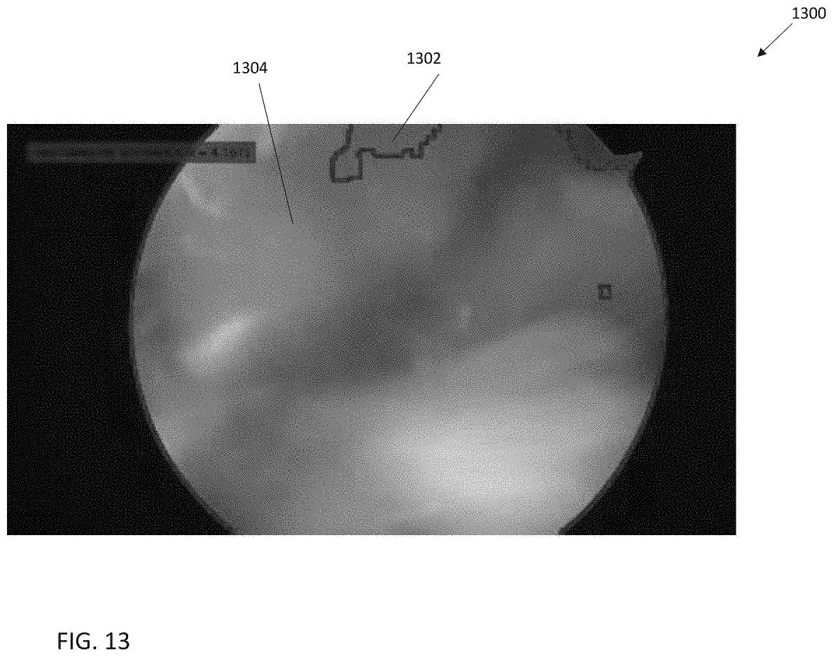

FIG. 13 illustrates an exemplary endoscopic image with segmented snowy area regions according to examples of the disclosure. In the example of FIG. 13, the image 1300 can include one or more snowy area regions 1304 as identified at step 1204 in the example of FIG. 12. In one or more examples, the snowy area regions can be distinguished from other regions 1302 where the snow globe effect is not present.

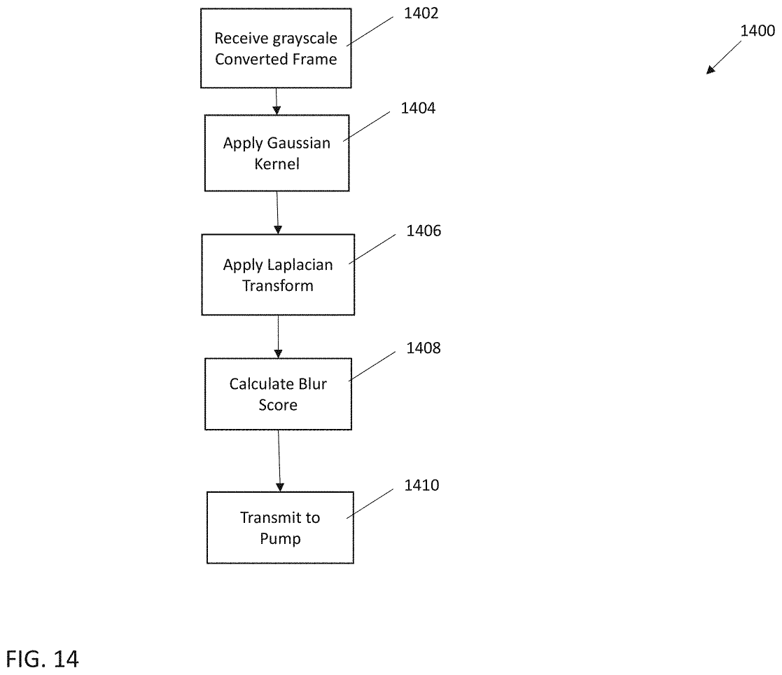

FIG. 14 illustrates an exemplary process for detecting turbidity in an image according to examples of the disclosure. In one or more examples, the process 1400 of FIG. 14 can begin at step 1402 wherein a grayscale converted image is received as described above with respect to step 714 of FIG. 7. Once the grayscale image is received at step 1402, the process 1400 can move to step 1404 wherein the image is convolved with a Gaussian kernel. Convolving the image with a Gaussian kernel at step 1404 can suppress the noise in the image, to allow for further image processing. Once the Gaussian kernel is applied at step 1404, the process 1400 can move to step 1406 wherein a Laplacian transform is applied to the image. The Laplacian transform can be used to find areas of rapid change (edges) in the image.

Once the Laplacian transform is applied at step 1406, the process 1400 can move to step 1408 wherein a blur score is calculated from the result of step 1406. In one or more examples, the blur score can represent the degree of blur in the image. A high blur score can indicate that the image is blurry and can therefor indicate the presence of turbidity in the image. A low blur score can indicate the absence of turbidity. Once the blur score has been calculated at step 1408, the process 1400 can move to step 1410 wherein the blur score is transmitted to the surgical pump or a controller communicatively coupled to the surgical pump.

The pressure or inflow/outflow settings of the surgical pump can be adjusted based on the calculated blur score. In one or more examples, the blur score calculated at step 1408 can be compared against a pre-determined threshold to determine if the pump needs to be adjusted based on the blur score. In one or more examples, if the blur score is higher than the pre-determined threshold then the pump can take action to increase the pressure (described in further detail below). The pre-determined threshold, in one or more examples, can be empirically determined. Additionally or alternatively, the pre-determined threshold can be set based on the surgeon's preferences. In one or more examples, the inflow of the pump can be pulsed to keep stagnant fluid away from the scope.

As described above, each of the individual clarity classifiers described above with respect to FIGS. 7-14 can individually cause the surgical pump to increase or decrease the pressure settings by increasing or decreasing the inflow/outflow or by increasing and decreasing the suction of the surgical pump. In one or more examples, the clarity classifiers can also collectively cause an adjustment to the surgical pump pressure settings.

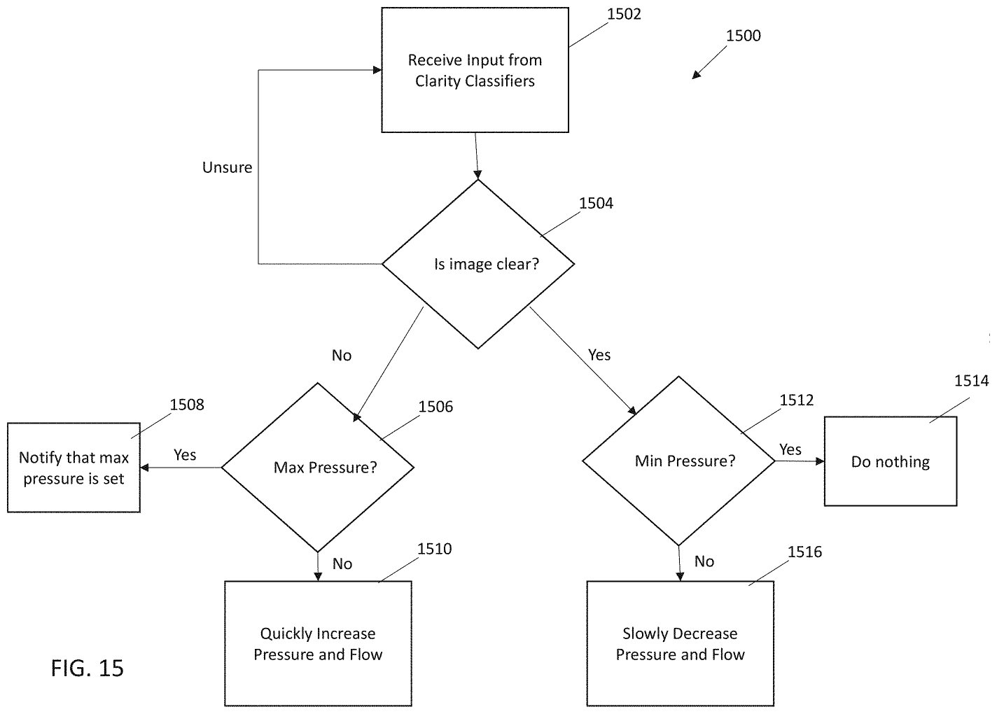

FIG. 15 illustrates an exemplary process for adjusting the settings of a surgical pump based on the image clarity according to examples of the disclosure. In one or more examples, the process 1500 of FIG. 15 can begin at step 1502 wherein the data from each clarity based classifier is received. The data can represent the output values of each classifier that is transmitted to the surgical pump or a controller communicatively coupled to the surgical pump as described above. Once the inputs are received at step 1502, the process 1500 can move to step 1504 wherein a determination is made as to whether the image is clear. As described above, the determination can be based on whether the outputs of the classifiers are greater than or less than a pre-determined threshold. In one or more examples, if one of the outputs of the classifiers is greater than its corresponding pre-determined threshold, then it can be determined that the image is not clear. In one or more examples, if a certain number of classifier outputs are higher than their corresponding pre-determined thresholds, then the process 1500 at step 1504 can determine that the image is not clear. In one or more examples, if a plurality of outputs are greater than their corresponding pre-determined thresholds, and a plurality of outputs are less than their corresponding pre-determined thresholds, then the process 1500 at step 1504 can determine that it is unsure about the clarity of the image.

In one or more examples, if the process 1500 at step 1504 determines that it is unsure about the image, then the process 1500 can do nothing with respect to the pressure settings of the surgical pump and revert back to step 1502 of process 1500 to receive further data from the one or more clarity based classifiers. A determination of unsure can mean that it is not apparent that there is a visual disturbance and so rather than change the settings of the pump, the process can instead do nothing and wait for more data.

In one or more examples, if the process 1500 at step 1504 determines that the image is not clear than the process 1500 can move to step 1506 wherein the process 1500 can determine if the surgical pump is at a maximum allowable pressure. As described above, if an image is not clear, then the pump may need to take one or more actions to increase the pressure in the surgical cavity so as to remove or minimize one or more visual disturbances that are causing the image to not be clear. However, as also described above, there exists a maximum pressure setting for the pump that if exceeded could cause injury or damage to the patient. This pressure level can be context dependent. For instance, the maximum allowable pressure for a knee surgery may be different than the maximum allowable pressure for a shoulder surgery. Thus, while a determination that the image is not clear may require the pressure exerted by the pump to increase, a check is first done at step 1506 to make sure that the pump is not already at its maximum allowable pressure settings for the area in which the surgery is occurring (or other factors that can influence the maximum allowable pressure). In one or more examples, if the process 1500 at step 1506 determines that the surgical pump is already at the maximum pressure, then the process 1500 can move to step 1508 wherein the surgeon is notified that the pump is at maximum pressure. In one or more examples, the notification may take the form of a visual display or audible tone that is configured to alert the surgeon that the image is not clear but that the pressure cannot be increased.

In one or more examples, if the process 1500 determines at step 1506 that the pump is not at max pressure, then the process 1500 at step 1506 can move to step 1510 wherein the pressure and/or flow of the pump is quickly increased in an attempt to clear or minimize visual disturbances in the surgical cavity. In one or more examples of the disclosure, the pressure exerted by the pump can be increased using a proportional-integral-derivative (PID) algorithm so as to increase the pressure in a controlled and accurate manner. In one or more examples, the pressure exerted by the pump can be increased using a Predictive Function Control (PFC) to control the increase or decrease in the pressure applied by the pump.

Referring back to step 1504, in one or more examples, if it is determined that the image is not clear, then the process 1500 can move to step 1512 wherein a determination is made as to whether the surgical pump is at its minimum allowable pressure setting. As described above, the goal of the surgical pump can be to apply the least amount of pressure to a surgical cavity as possible so as to minimize the risk of damage or injury to the patient. Thus, in one or more examples, in addition to increasing pressure to remove visual disturbances, the process 1500 can be configured to decrease the pressure in the joint, if it is determined that there are no visual disturbances and the image is clear. A determination that the image is clear can present an opportunity for the surgical pump to reduce the pressure (because it may not be needed). Thus, at step 1512, if it is determined that the device is already at the minimum pressure needed, then the process 1500 can move to step 1514 wherein the surgical pump is not adjusted. This pressure level can be context dependent. For instance, the minimum allowable pressure for a knee surgery may be different than the minimum allowable pressure for a shoulder surgery. If, however, a determination is made that the surgical pump is not at its minimum setting at step 1512, then the process 1500 can move to step 1516 wherein the pressure exerted by the surgical pump can be reduced. In one or more examples of the disclosure, the pressure exerted by the pump can be decreased using a PID algorithm so as to decrease the pressure in a controlled and accurate manner.

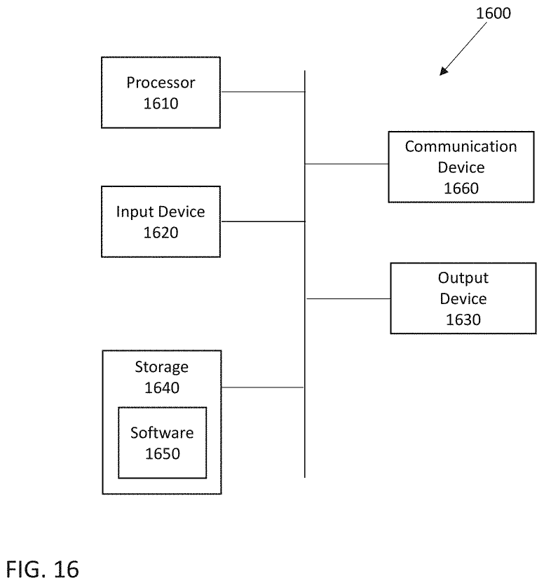

FIG. 16 illustrates an example of a computing system 1600, in accordance with some examples, that can be used for one or more components of system 100 of FIG. 1, such as one or more components of camera head 108, camera control unit 112, and image processing unit 116. System 1600 can be a computer connected to a network, such as one or more networks of hospital, including a local area network within a room of a medical facility and a network linking different portions of the medical facility. System 1600 can be a client or a server. As shown in FIG. 16, system 1600 can be any suitable type of processor-based system, such as a personal computer, workstation, server, handheld computing device (portable electronic device) such as a phone or tablet, or dedicated device. The system 1600 can include, for example, one or more of input device 1620, output device 1630, one or more processors 1610, storage 1640, and communication device 1660. Input device 1620 and output device 1630 can generally correspond to those described above and can either be connectable or integrated with the computer.

Input device 1620 can be any suitable device that provides input, such as a touch screen, keyboard or keypad, mouse, gesture recognition component of a virtual/augmented reality system, or voice-recognition device. Output device 1630 can be or include any suitable device that provides output, such as a display, touch screen, haptics device, virtual/augmented reality display, or speaker.

Storage 1640 can be any suitable device that provides storage, such as an electrical, magnetic, or optical memory including a RAM, cache, hard drive, removable storage disk, or other non-transitory computer readable medium. Communication device 1660 can include any suitable device capable of transmitting and receiving signals over a network, such as a network interface chip or device. The components of the computing system 1600 can be connected in any suitable manner, such as via a physical bus or wirelessly.

Processor(s) 1610 can be any suitable processor or combination of processors, including any of, or any combination of, a central processing unit (CPU), field programmable gate array (FPGA), application-specific integrated circuit (ASIC), and a graphical processing unit (GPU). Software 1650, which can be stored in storage 1640 and executed by one or more processors 1610, can include, for example, the programming that embodies the functionality or portions of the functionality of the present disclosure (e.g., as embodied in the devices as described above).

Software 1650 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1640, that can contain or store programming for use by or in connection with an instruction execution system, apparatus, or device.

Software 1650 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport computer readable medium can include, but is not limited to, an electronic, magnetic, optical, electromagnetic, or infrared wired or wireless propagation medium.

System 1600 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

System 1600 can implement any operating system suitable for operating on the network. Software 1650 can be written in any suitable programming language, such as C, C++, Java, or Python. In various examples, application software embodying the functionality of the present disclosure can be deployed in different configurations, such as in a client/server arrangement or through a Web browser as a Web-based application or Web service, for example.

The foregoing description, for the purpose of explanation, has been described with reference to specific examples. However, the illustrative discussions above are not intended to be exhaustive or to limit the invention to the precise forms disclosed. Many modifications and variations are possible in view of the above teachings. The examples were chosen and described in order to best explain the principles of the techniques and their practical applications. Others skilled in the art are thereby enabled to best utilize the techniques and various examples with various modifications as are suited to the particular use contemplated. For the purpose of clarity and a concise description, features are described herein as part of the same or separate examples; however, it will be appreciated that the scope of the disclosure includes examples having combinations of all or some of the features described.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A method for controlling a fluid pump for use in surgical procedures, the method comprising:
receiving video data captured from an imaging tool configured to image an internal portion of a patient;
applying one or more machine learning classifiers to the received video data to generate one or more classification metrics based on the received video data, wherein the one or more machine learning classifiers are created using a supervised training process that comprises using one or more annotated images to train the machine learning classifier;
determining the presence of one or more conditions in the received video data based on the generated one or more classification metrics; and
determining an adjusted setting for the flow through or head pressure from the fluid pump based on the determined presence of the one or more conditions in the received video data.

2. The method of claim 1, wherein the one or more machine learning classifiers comprises a joint type machine learning classifier configured to generate one or more classification metrics associated with identifying a type of joint pictured in the received video data.

3. The method of claim 2, wherein the joint type machine learning classifier is configured to identify one or more joints selected from the group consisting of a hip, a shoulder, a knee, an ankle, a wrist, and an elbow.

4. The method of claim 3, wherein the joint type machine learning classifier is configured to generate one or more classification metrics associated with identifying whether the imaging tool is not within a joint.

5. The method of claim 4, wherein the one or more machine learning classifiers include a procedure stage machine learning classifier configured to generate one or more classification metrics associated with identifying a procedure stage being performed in the received video data.

6. The method of claim 1, wherein the one or more machine learning classifiers comprises an instrument identification machine classifier configured to generate one or more classification metrics associated with identifying one or more instruments in the received video data.

7. The method of claim 6, wherein the instrument identification machine classifier is configured to identify instruments selected from the group consisting of a shaver tool, a radio frequency (RF) probe, and a dedicated suction device.

8. The method of claim 6, wherein the fluid pump is configured to activate a suction functionality of the one or more instruments based on the one or more classification metrics generated by the instrument identification machine classifier.

9. The method of claim 1, wherein the one or more machine learning classifiers include an image clarity machine learning classifier configured to generate one or more classification metrics associated with a clarity of the received video data.

10. The method of claim 9, wherein the image clarity machine classifier is configured to generate one or more classification metrics associated with an amount of blood visible in the received video data.

11. The method of claim 9, wherein the image clarity machine classifier is configured to generate one or more classification metrics associated with an amount of bubbles visible in the received video data.

12. The method of claim 9, wherein the image clarity machine classifier is configured to generate one or more classification metrics associated with an amount of debris visible in the received video data.

13. The method of claim 9, wherein determining the presence of one or more conditions in the received video data based on the generated one or more classification metrics comprises determining if a clarity of the video is above a pre-determined threshold, and wherein the determination is based on the one more classification metrics generated by the image clarity machine classifier.

14. A system for controlling a fluid pump for use in surgical procedures, the system comprising:
a memory;
one or more processors;
wherein the memory stores one or more programs that when executed by the one or more processors, cause the one or more processors to:
receive video data captured from an imaging tool configured to image an internal portion of a patient;
apply one or more machine learning classifiers to the received video data to generate one or more classification metrics based on the received video data, wherein the one or more machine learning classifiers are created using a supervised training process that comprises using one or more annotated images to train the machine learning classifier;
determine the presence of one or more conditions in the received video data based on the generated one or more classification metrics; and
adjust the flow through or head pressure from the fluid pump based on the determined presence of the one or more conditions in the received video data.

15. The system of claim 14, wherein the one or more machine learning classifiers comprises a joint type machine learning classifier configured to generate one or more classification metrics associated with identifying a type of joint pictured in the received video data.

16. The system of claim 15, wherein the joint type machine learning classifier is configured to identify one or more joints selected from the group consisting of a hip, a shoulder, a knee, an ankle, a wrist, and an elbow.

17. The system of claim 16, wherein the joint type machine learning classifier is configured to generate one or more classification metrics associated with identifying whether the imaging tool is not within a joint.

18. The system of claim 17, wherein the one or more machine learning classifiers include a procedure stage machine learning classifier configured to generate one or more classification metrics associated with identifying a procedure stage being performed in the received video data.

19. The system of claim 14, wherein the one or more machine learning classifiers comprises an instrument identification machine classifier configured to generate one or more classification metrics associated with identifying one or more instruments in the received video data.

20. The system of claim 19, wherein the instrument identification machine classifier is configured to identify instruments selected from the group consisting of a shaver tool, a radio frequency (RF) probe, and a dedicated suction device.

21. The system of claim 19, wherein the fluid pump is configured to activate a suction functionality of the one or more instruments based on the one or more classification metrics generated by the instrument identification machine classifier.

22. The system of claim 14, wherein the one or more machine learning classifiers include an image clarity machine learning classifier configured to generate one or more classification metrics associated with a clarity of the received video data.

23. The system of claim 22, wherein the image clarity machine classifier is configured to generate one or more classification metrics associated with an amount of blood visible in the received video data.

24. The system of claim 22, wherein the image clarity machine classifier is configured to generate one or more classification metrics associated with an amount of bubbles visible in the received video data.

25. The system of claim 22, wherein the image clarity machine classifier is configured to generate one or more classification metrics associated with an amount of debris visible in the received video data.

26. The system of claim 22, wherein determining the presence of one or more conditions in the received video data based on the generated one or more classification metrics comprises determining if a clarity of the video is above a pre-determined threshold, and wherein the determination is based on the one more classification metrics generated by the image clarity machine classifier.

27. A non-transitory computer readable storage medium storing one or more programs for controlling a fluid pump for use in surgical procedures, for execution by one or more processors of an electronic device that when executed by the device, cause the device to:
receive video data captured from an imaging tool configured to image an internal portion of a patient;
apply one or more machine learning classifiers to the received video data to generate one or more classification metrics based on the received video data, wherein the one or more machine learning classifiers are created using a supervised training process that comprises using one or more annotated images to train the machine learning classifier;
determine the presence of one or more conditions in the received video data based on the generated one or more classification metrics; and
adjust the flow through or head pressure from the fluid pump based on the determined presence of the one or more conditions in the received video data.

28. The non-transitory computer readable storage medium of claim 27, wherein the one or more machine learning classifiers comprises a joint type machine learning classifier configured to generate one or more classification metrics associated with identifying a type of joint pictured in the received video data.

29. The non-transitory computer readable storage medium of claim 28, wherein the joint type machine learning classifier is configured to identify one or more joints selected from the group consisting of a hip, a shoulder, a knee, an ankle, a wrist, and an elbow.

30. The non-transitory computer readable storage medium of claim 29, wherein the joint type machine learning classifier is configured to generate one or more classification metrics associated with identifying whether the imaging tool is not within a joint.

31. The non-transitory computer readable storage medium of claim 30, wherein the one or more machine learning classifiers include a procedure stage machine learning classifier configured to generate one or more classification metrics associated with identifying a procedure stage being performed in the received video data.

32. The non-transitory computer readable storage medium of claim 27, wherein the one or more machine learning classifiers comprises an instrument identification machine classifier configured to generate one or more classification metrics associated with identifying one or more instruments in the received video data.

33. The non-transitory computer readable storage medium of claim 32, wherein the instrument identification machine classifier is configured to identify instruments selected from the group consisting of a shaver tool, a radio frequency (RF) probe, and a dedicated suction device.

34. The non-transitory computer readable storage medium of claim 32, wherein the fluid pump is configured to activate a suction functionality of the one or more instruments based on the one or more classification metrics generated by the instrument identification machine classifier.

35. The non-transitory computer readable storage medium of claim 27, wherein the one or more machine learning classifiers include an image clarity machine learning classifier configured to generate one or more classification metrics associated with a clarity of the received video data.

36. The non-transitory computer readable storage medium of claim 35, wherein the image clarity machine classifier is configured to generate one or more classification metrics associated with an amount of blood visible in the received video data.

37. The non-transitory computer readable storage medium of claim 35, wherein the image clarity machine classifier is configured to generate one or more classification metrics associated with an amount of bubbles visible in the received video data.

38. The s non-transitory computer readable storage medium of claim 35, wherein the image clarity machine classifier is configured to generate one or more classification metrics associated with an amount of debris visible in the received video data.

39. The non-transitory computer readable storage medium of claim 35, wherein determining the presence of one or more conditions in the received video data based on the generated one or more classification metrics comprises determining if a clarity of the video is above a pre-determined threshold, and wherein the determination is based on the one more classification metrics generated by the image clarity machine classifier.
